(19) United States
(12) Patent Application Publication
(10) Pub. No.: US 2013/0217113 A1
(43) Pub. Date: Aug. 22, 2013

(54) SYSTEM FOR AND METHODS OF PROMOTING CELL LYSIS IN DROPLET ACTUATORS

(71) Applicants: Vijay Srinivasan, Durham, NC (US); William Craig Bauer, Raleigh, NC (US); Gregory F. Smith, Cary, NC (US); Ryan A. Sturmer, Durham, NC (US); Zhishan Hua, Greensboro, NC (US); Allen E. Eckhardt, Durham, NC (US)

(72) Inventors: Vijay Srinivasan, Durham, NC (US); William Craig Bauer, Raleigh, NC (US); Gregory F. Smith, Cary, NC (US); Ryan A. Sturmer, Durham, NC (US); Zhishan Hua, Greensboro, NC (US); Allen E. Eckhardt, Durham, NC (US)

(73) Assignee: ADVANCED LIQUID LOGIC INC., Research Triangle Park, NC (US)

(21) Appl. No.: 13/809,762

(22) PCT Filed: Jul. 12, 2011
(86) PCT No.: PCT/US11/43650

§ 371 Date: Jan. 22, 2013

Related U.S. Application Data
(60) Provisional application No. 61/364,645, filed on Jul. 15, 2010.

(51) Int. Cl. C12M 1/42

(52) U.S. Cl. CPC C12M 1/42
USPC 435/306.1

(57) ABSTRACT

The invention relates to a droplet actuator for conducting droplet operations. The actuator includes a bottom substrate and a top substrate separated from the bottom substrate to form a gap. An arrangement of droplet operations electrodes may be located on a surface of the bottom substrate and/or top substrate. Optionally, a sample reservoir may hold a quantity of a sample fluid containing cells. A disruption device which can take various forms is used to lyse the cells in the sample or in a sample droplet to thereby conduct operations on samples having lysed cells therein.

### 1 RELATED APPLICATIONS

In addition to the patent applications cited herein, each of which is incorporated herein by reference, this patent application is related to and claims priority to U.S. Provisional Patent Application No. 61/364,645, entitled “Systems for and Methods of Promoting Cell Lysis in Droplet Actuators,” filed on Jul. 15, 2010. The entire disclosure of which is incorporated herein by reference.

This patent application is related to U.S. Provisional Patent Application Nos. 61/314,835, entitled “Systems for and Methods of Promoting Cell Lysis in Droplet Actuators,” filed on Mar. 17, 2010; and 61/317,999, entitled “Systems for and Methods of Promoting Cell Lysis in Droplet Actuators,” filed on Mar. 26, 2010, each of which is incorporated herein by reference.

### 2 FIELD OF THE INVENTION

The present invention generally relates to a droplet actuator for conducting droplet operations. In particular, the present invention is directed to a droplet actuator which includes devices for lysing cells in a sample fluid to create a lysate for conducting droplet operations on droplets formed from the lysate.

### 3 BACKGROUND OF THE INVENTION

A droplet actuator typically includes one or more substrates configured to form a surface or gap for conducting droplet operations. The one or more substrates include electrodes and establish a droplet operations surface or gap for conducting droplet operations. The droplet operations substrate or the gap between the substrates may be coated or filled with a filler fluid that is immiscible with the liquid that forms the droplets. Droplet operations are controlled by the electrodes. Certain assay protocols require disruption of materials, such as tissues, cells or spores. There is a need for techniques for disruption of such materials in a droplet actuator system, e.g., to provide a complete sample-to-answer system for analyses that require cell or spore lysis.

### 4 BRIEF DESCRIPTION OF THE INVENTION

The present invention is directed to a droplet actuator for conducting droplet operations. A bottom substrate and a top substrate are separated from each other to form a gap. Droplet operations electrodes are arranged on at least one of the bottom and top substrate for conducting droplet operation. A disruption device is associated with the droplet actuator for lysing cells in a cell-containing a sample on which droplet operations are to be conducted.

In one embodiment, a droplet actuator for conducting droplet operations is provided. The droplet actuator may include, a bottom substrate and a top substrate separated from the bottom substrate to form a gap; an arrangement of droplet operations electrodes on at least one of the bottom and top substrate for conducting droplet operations; a sample reservoir for holding a quantity of sample fluid containing cells to be lysed; and a sonication device associated with the sample reservoir for lysing cells in a sample fluid therein to create a lysate.

In another embodiment, a droplet actuator for conducting droplet operations is provided. The droplet actuator may include, a bottom substrate and a top substrate separated from the bottom substrate to form a gap; an arrangement of droplet operations electrodes on at least one of the bottom and top substrate for conducting droplet operations; a sample supply for supplying a quantity of sample fluid containing cells to be lysed into the gap; and a sonication device for lysing cells in the sample in the sample reservoir or in droplets in the gap to conduct droplet operations thereon.

In yet another embodiment a droplet actuator for conducting droplet operations is provided. The droplet actuator may include, a bottom substrate and a top substrate separated from the bottom substrate to form a gap; an arrangement of droplet operations electrodes on at least one of the bottom and top substrate for conducting droplet operations; a sample supply for supplying a quantity of sample fluid containing cells to be lysed into the gap; and a heating device associated with the droplet actuator for causing lysis of cells in a sample fluid.

In still yet another embodiment a droplet actuator for conducting droplet operations is provided. The droplet actuator may include, a bottom substrate and a top substrate separated from the bottom substrate to form a gap; an arrangement of droplet operations electrodes on at least one of the bottom and top substrate for conducting droplet operations; a sample supply for supplying a quantity of sample fluid containing cells to be lysed into the gap; and a cell disruption device for lysing cells in a sample fluid.

In still yet another embodiment, a sample reservoir may be provided for holding a quantity of sample fluid containing cells to be lysed. The disruption device may be associated with the sample reservoir, or maybe part of and integral with the droplet actuator for conducting disruption of cells in sample cell containing droplets within the gap.

In still yet another embodiment, the disruption device may be a sonication device, more typically an ultrasonic actuator, which may be used to apply ultrasonic energy to the sample reservoir or to the droplets in the gap. In an alternative aspect, the device may be a zirconate titanate actuator.

In still yet another embodiment, thermal energy may be provided to cell-containing sample fluids in the form of a heater, laser, or other suitable thermal means.

In still yet another embodiment, mechanical disruption to cause application of shear on the cells may be used to lyse the cells. Yet still further, magnetic beads may also be employed and activated through an inductor device, and an electromagnet, or other like devices within a sample droplet to cause physical disruption of the cells. Yet still further, the disruption device may be an ultrasonic device, or exciting particles in cell-containing sample droplets. Alternatively, electrodes may be used for creating an electric field which disrupts cells in the sample.

These and other features are described in greater detail in the following Detailed Description made with reference to the appended drawings.

### 5 DEFINITIONS

As used herein, the following terms have the meanings indicated.

“Activate,” with reference to one or more electrodes, means affecting a change in the electrical state of the one or more electrodes which, in the presence of a droplet, results in a droplet operation. Activation of an electrode can be accomplished using alternating or direct current. Any suitable voltage may be used. For example, an electrode may be activated using a voltage which is greater than about 150 V, or greater than about 200 V, or greater than about 250 V, or from about 275 V to about 375 V, or about 300 V. Where alternating current is used, any suitable frequency may be employed. For example, an electrode may be activated using alternating current having a frequency from about 1 Hz to about 100 Hz, or from about 10 Hz to about 60 Hz, or from about 20 Hz to about 40 Hz, or about 30 Hz.

“Bead,” with respect to beads on a droplet actuator, means any bead or particle that is capable of interacting with a droplet on or in proximity with a droplet actuator. Beads may be any of a wide variety of shapes, such as spherical, generally spherical, egg shaped, disc shaped, cubical, amorphous and other three dimensional shapes. The bead may, for example, be capable of being subjected to a droplet operation in a droplet on a droplet actuator or otherwise configured with respect to a droplet actuator in a manner which permits a droplet on the droplet actuator to be brought into contact with the bead on the droplet actuator and/or off the droplet actuator. Beads may be provided in a droplet, in a droplet operations gap, or on a droplet operations surface. Beads may be provided in a reservoir that is external to a droplet operations gap or situated apart from a droplet operations surface, and the reservoir may be associated with a fluid path that permits a droplet including the beads to be brought into a droplet operations gap or into contact with a droplet operations surface. Beads may be manufactured using a wide variety of materials, including for example, resins, and polymers. The beads may be any suitable size, including for example, microbeads, microparticles, nanobeads and nanoparticles. In some cases, beads are magnetically responsive; in other cases beads are not significantly magnetically responsive. For magnetically responsive beads, the magnetically responsive material may constitute substantially all of a bead, a portion of a bead, or only one component of a bead. The remainder of the bead may include, among other things, polymeric material, coatings, and moieties which permit attachment of an assay reagent. Examples of suitable beads include flow cytometry microbeads, polystyrene microparticles and nanoparticles, functionalized polystyrene microparticles and nanoparticles, coated polystyrene microparticles and nanoparticles, silica microbeads, fluorescent microspheres and nanospheres, functionalized fluorescent microspheres and nanospheres, coated fluorescent microspheres and nanospheres, color dyed microparticles and nanoparticles, magnetic microparticles and nanoparticles, superparamagnetic microparticles and nanoparticles (e.g., DYNABEADS® particles, available from Invitrogen Group, Carlsbad, Calif.), fluorescent microparticles and nanoparticles, coated magnetic microparticles and nanoparticles, ferromagnetic microparticles and nanoparticles, coated ferromagnetic microparticles and nanoparticles, and those described in U.S. Patent Publication Nos. 20050260686, entitled “Multiplex flow assays preferably with magnetic particles as solid phase,” published on Nov. 24, 2005; 20030132538, entitled “Encapsulation of discrete quanta of fluorescent particles,” published on Jul. 17, 2003; 20050118574, entitled “Multiplexed Analysis of Clinical Specimens Apparatus and Method,” published on Jun. 2, 2005; 20050277197. Entitled “Microparticles with Multiple Fluorescent Signals and Methods of Using Same,” published on Dec. 15, 2005; 20060159962, entitled “Magnetic Microspheres for use in Fluorescence-based Applications,” published on Jul. 20, 2006; the entire disclosures of which are incorporated herein by reference for their teaching concerning beads and magnetically responsive materials and beads. Beads may be pre-coupled with a biomolecule or other substance that is able to bind to and form a complex with a biomolecule. Beads may be pre-coupled with an antibody, protein or antigen, DNA/RNA probe or any other molecule with an affinity for a desired target. Examples of droplet actuator techniques for immobilizing magnetically responsive beads and/or non-magnetically responsive beads and/or conducting droplet operations protocols using beads are described in U.S. patent application Ser. No. 11/639,566, entitled “Droplet-Based Particle Sorting,” filed on Dec. 15, 2006; U.S. Patent Application No. 61/039,183, entitled “Multiplexing Bead Detection in a Single Droplet,” filed on Mar. 25, 2008; U.S. Patent Application No. 61/047,789, entitled “Droplet Actuator Devices and Droplet Operations Using Beads,” filed on Apr. 25, 2008; U.S. Patent Application No. 61/086,183, entitled “Droplet Actuator Devices and Methods for Manipulating Beads,” filed on Aug. 5, 2008; International Patent Application No. PCT/US2008/053545, entitled “Droplet Actuator Devices and Methods Employing Magnetic Beads,” filed on Feb. 11, 2008; International Patent Application No. PCT/US2008/058018, entitled “Bead-based Multiplexed Analytical Methods and Instrumentation,” filed on Mar. 24, 2008; International Patent Application No. PCT/US2008/058047, “Bead Sorting on a Droplet Actuator,” filed on Mar. 23, 2008; and International Patent Application No. PCT/US2006/047486, entitled “Droplet-based Biochemistry,” filed on Dec. 11, 2006; the entire disclosures of which are incorporated herein by reference. Bead characteristics may be employed in the multiplexing aspects of the invention. Examples of beads having characteristics suitable for multiplexing, as well as methods of detecting and analyzing signals emitted from such beads, may be found in U.S. Patent Publication No. 20080305481, entitled “Systems and Methods for Multiplex Analysis of PCR in Real Time,” published on Dec. 11, 2008; U.S. Patent Publication No. 20080151240, “Methods and Systems for Dynamic Range Expansion,” published on Jun. 26, 2008; U.S. Patent Publication No. 20070207513, entitled “Methods, Products, and Kits for Identifying an Analyte in a Sample,” published on Sep. 6, 2007; U.S. Patent Publication No. 20070064990, entitled “Methods and Systems for Image Data Processing,” published on Mar. 22, 2007; U.S. Patent Publication No. 20060159962, entitled “Magnetic Microspheres for use in Fluorescence-based Applications,” published on Jul. 20, 2006; U.S. Patent Publication No. 20050277197, entitled “Microparticles with Multiple Fluorescent Signals and Methods of Using Same,” published on Dec. 15, 2005; and U.S. Patent Publication No. 20050118574, entitled “Multiplexed Analysis of Clinical Specimens Apparatus and Method,” published on Jun. 2, 2005.

“Droplet” means a volume of liquid on a droplet actuator. Typically, a droplet is at least partially bounded by a filler fluid. For example, a droplet may be completely surrounded by a filler fluid or may be bounded by filler fluid and one or more surfaces of the droplet actuator. As another example, a droplet may be bounded by filler fluid, one or more surfaces of the droplet actuator, and/or the atmosphere. As yet another example, a droplet may be bounded by filler fluid and the atmosphere. Droplets may, for example, be aqueous or non-aqueous or may be mixtures or emulsions including aqueous and non-aqueous components. Droplets may take a wide variety of shapes; nonlimiting examples include generally disc shaped, slug shaped, truncated sphere, ellipsoid, spherical, partially compressed sphere, hemispherical, ovoid, cylindrical, combinations of such shapes, and various shapes formed during droplet operations, such as merging or splitting or formed as a result of contact of such shapes with one or more surfaces of a droplet actuator. For examples of droplet fluids that may be subjected to droplet operations using the approach of the invention, see International Patent Application No. PCT/US 06/47486, entitled, “Droplet-Based Biochemistry,” filed on Dec. 11, 2006. In various embodiments, a droplet may include a biological sample, such as whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs and biological washes. Moreover, a droplet may include a reagent, such as water, deionized water, saline solutions, acidic solutions, basic solutions, detergent solutions and/or buffers. Other examples of droplet contents include reagents, such as a reagent for a biochemical protocol, such as a nucleic acid amplification protocol, an affinity-based assay protocol, an enzymatic assay protocol, a sequencing protocol, and/or a protocol for analyses of biological fluids.

“Droplet Actuator” means a device for manipulating droplets. For examples of droplet actuators, see Pamula et al., U.S. Pat. No. 6,911,132, entitled “Apparatus for Manipulating Droplets by Electrowetting-Based Techniques,” issued on Jun. 28, 2005; Pamula et al., U.S. patent application Ser. No. 11/343,284, entitled “Apparatuses and Methods for Manipulating Droplets on a Printed Circuit Board,” filed on filed on Jan. 30, 2006; Pollack et al., International Patent Application No. PCT/US2006/047486, entitled “Droplet-Based Biochemistry,” filed on Dec. 11, 2006; Shenderov, U.S. Pat. Nos. 6,773,566, entitled “Electrostatic Actuators for Microfluidics and Methods for Using Same,” issued on Aug. 10, 2004 and 6,565,727, entitled “Actuators for Microfluidics Without Moving Parts,” issued on Jan. 24, 2000; Kim and/or Shah et al., U.S. patent application Ser. Nos. 10/343,261, entitled “Electrowetting-driven Micropumping,” filed on Jan. 27, 2003, 11/275,668, entitled “Method and Apparatus for Promoting the Complete Transfer of Liquid Drops from a Nozzle,” filed on Jan. 23, 2006, 11/460,188, entitled “Small Object Moving on Printed Circuit Board,” filed on Jan. 23, 2006, 12/465,935, entitled “Method for Using Magnetic Particles in Droplet Microfluidics,” filed on May 14, 2009, and 12/513,157, entitled “Method and Apparatus for Real-time Feedback Control of Electrical Manipulation of Droplets on Chip,” filed on Apr. 30, 2009; Velev, U.S. Pat. No. 7,547,380, entitled “Droplet Transportation Devices and Methods Having a Fluid Surface,” issued on Jun. 16, 2009; Sterling et al., U.S. Pat. No. 7,163,612, entitled “Method, Apparatus and Article for Microfluidic Control via Electrowetting, for Chemical, Biochemical and Biological Assays and the Like,” issued on Jan. 16, 2007; Becker and Gascoyne et al., U.S. Pat. Nos. 7,641,779, entitled “Method and Apparatus for Programmable fluidic Processing,” issued on Jan. 5, 2010, and 6,977,033, entitled “Method and Apparatus for Programmable fluidic Processing,” issued on Dec. 20, 2005; Decre et al., U.S. Pat. No. 7,328,979, entitled “System for Manipulation of a Body of Fluid,” issued on Feb. 12, 2008; Yamakawa et al., U.S. Patent Pub. No. 20060039823, entitled “Chemical Analysis Apparatus,” published on Feb. 23, 2006; Wu, International Patent Pub. No. WO/2009/003184, entitled “Digital Microfluidics Based Apparatus for Heat-exchanging Chemical Processes,” published on Dec. 31, 2008; Fouillet et al., U.S. Patent Pub. No. 20090192044, entitled “Electrode Addressing Method,” published on Jul. 30, 2009; Fouillet et al., U.S. Pat. No. 7,052,244, entitled “Device for Displacement of Small Liquid Volumes Along a Micro-catenary Line by Electrostatic Forces,” issued on May 30, 2006; Marchand et al., U.S. Patent Pub. No. 20080124252, entitled “Droplet Microreactor,” published on May 29, 2008; Adachi et al., U.S. Patent Pub. No. 20090321262, entitled “Liquid Transfer Device,” published on Dec. 31, 2009; Roux et al., U.S. Patent Pub. No. 20050179746, entitled “Device for Controlling the Displacement of a Drop Between two or Several Solid Substrates,” published on Aug. 18, 2005; Dhindsa et al., “Virtual Electrowetting Channels Electronic Liquid Transport with Continuous Channel Functionality,” Lab Chip, 10:832-836 (2010); the entire disclosures of which are incorporated herein by reference, along with their priority documents. Certain droplet actuators will include one or more substrates arranged with a gap therebetween and electrodes associated with (e.g., layered on, attached to, and/or embedded in) the one or more substrates and arranged to conduct one or more droplet operations. For example, certain droplet actuators will include a base (or bottom) substrate, droplet operations electrodes associated with the substrate, one or more dielectric layers atop the substrate and/or electrodes, and optionally one or more hydrophobic layers atop the substrate, dielectric layers and/or the electrodes forming a droplet operations surface. A top substrate may also be provided, which is separated from the droplet operations surface by a gap, commonly referred to as a droplet operations gap. Various electrode arrangements on the top and/or bottom substrates are discussed in the above-referenced patents and applications and certain novel electrode arrangements are discussed in the description of the invention. During droplet operations it is preferred that droplets remain in continuous contact or frequent contact with a ground or reference electrode. A ground or reference electrode may be associated with the top substrate facing the gap, the bottom substrate facing the gap, in the gap. Where electrodes are provided on both substrates, electrical contacts for coupling the electrodes to a droplet actuator instrument for controlling or monitoring the electrodes may be associated with one or both plates. In some cases, electrodes on one substrate are electrically coupled to the other substrate so that only one substrate is in contact with the droplet actuator. In one embodiment, a conductive material (e.g., an epoxy, such as MASTER BOND™ Polymer System EP79, available from Master Bond, Inc., Hackensack, N.J.) provides the electrical connection between electrodes on one substrate and electrical paths on the other substrates, e.g., a ground electrode on a top substrate may be coupled to an electrical path on a bottom substrate by such a conductive material. Where multiple substrates are used, a spacer may be provided between the substrates to determine the height of the gap therebetween and define dispensing reservoirs. The spacer height may, for example, be from about 5 μm to about 600 μm, or about 100 μm to about 400 μm, or about 200 μm to about 350 μm, or about 250 μm to about 300 μm, or about 275 μm. The spacer may, for example, be formed of a layer of projections form the top or bottom substrates, and/or a material inserted between the top and bottom substrates. One or more openings may be provided in the one or more substrates for forming a fluid path through which liquid may be delivered into the droplet operations gap. The one or more openings may in some cases be aligned for interaction with one or more electrodes, e.g., aligned such that liquid flowed through the opening will come into sufficient proximity with one or more droplet operations electrodes to permit a droplet operation to be effected by the droplet operations electrodes using the liquid. The base (or bottom) and top substrates may in some cases be formed as one integral component. One or more reference electrodes may be provided on the base (or bottom) and/or top substrates and/or in the gap. Examples of reference electrode arrangements are provided in the above referenced patents and patent applications. In various embodiments, the manipulation of droplets by a droplet actuator may be electrode mediated, e.g., electrowetting mediated or dielectrophoresis mediated or Coulombic force mediated. Examples of other techniques for controlling droplet operations that may be used in the droplet actuators of the invention include using devices that induce hydrodynamic fluidic pressure, such as those that operate on the basis of mechanical principles (e.g. external syringe pumps, pneumatic membrane pumps, vibrating membrane pumps, vacuum devices, centrifugal forces, piezoelectric/ultrasonic pumps and acoustic forces); electrical or magnetic principles (e.g. electroosmotic flow, electrokinetic pumps, ferrofluidic plugs, electrohydrodynamic pumps, attraction or repulsion using magnetic forces and magnetohydrodynamic pumps); thermodynamic principles (e.g. gas bubble generation/phase-change-induced volume expansion); other kinds of surface-wetting principles (e.g. electrowetting, and optoelectrowetting, as well as chemically, thermally, structurally and radioactively induced surface-tension gradients); gravity; surface tension (e.g., capillary action); electrostatic forces (e.g., electroosmotic flow); centrifugal flow (substrate disposed on a compact disc and rotated); magnetic forces (e.g., oscillating ions causes flow); magnetohydrodynamic forces; and vacuum or pressure differential. In certain embodiments, combinations of two or more of the foregoing techniques may be employed to conduct a droplet operation in a droplet actuator of the invention. Similarly, one or more of the foregoing may be used to deliver liquid into a droplet operations gap, e.g., from a reservoir in another device or from an external reservoir of the droplet actuator (e.g., a reservoir associated with a droplet actuator substrate and a fluid path from the reservoir into the droplet operations gap). Droplet operations surfaces of certain droplet actuators of the invention may be made from hydrophobic materials or may be coated or treated to make them hydrophobic. For example, in some cases some portion or all of the droplet operations surfaces may be derivatized with low surface-energy materials or chemistries, e.g., by deposition or using in situ synthesis using compounds such as poly- or per-fluorinated compounds in solution or polymerizable monomers. Examples include TEFLON® AF (available from DuPont, Wilmington, Del.), members of the cytop family of materials, coatings in the FLUOROPEL® family of hydrophobic and superhydrophobic coatings (available from Cytonix Corporation, Beltsville, Md.), silane coatings, fluorosilane coatings, hydrophobic phosphonate derivatives (e.g., those sold by Aculon, Inc), and NOVEC™ electronic coatings (available from 3M Company, St. Paul, Minn.), and other fluorinated monomers for plasma-enhanced chemical vapor deposition (PECVD). In some cases, the droplet operations surface may include a hydrophobic coating having a thickness ranging from about 10 nm to about 1,000 nm. Moreover, in some embodiments, the top substrate of the droplet actuator includes an electrically conducting organic polymer, which is then coated with a hydrophobic coating or otherwise treated to make the droplet operations surface hydrophobic. For example, the electrically conducting organic polymer that is deposited onto a plastic substrate may be poly(3,4-ethylenedioxythiophene) poly(styrenesulfonate) (PEDOT:PSS). Other examples of electrically conducting organic polymers and alternative conductive layers are described in Pollack et al., International Patent Application No. PCT/US2010/040705, entitled “Droplet Actuator Devices and Methods,” the entire disclosure of which is incorporated herein by reference. One or both substrates may be fabricated using a printed circuit board (PCB), glass, indium tin oxide (ITO)-coated glass, and/or semiconductor materials as the substrate. When the substrate is ITO-coated glass, the ITO coating is preferably a thickness in the range of about 20 to about 200 nm, preferably about 50 to about 150 nm, or about 75 to about 125 nm, or about 100 nm. In some cases, the top and/or bottom substrate includes a PCB substrate that is coated with a dielectric, such as a polyimide dielectric, which may in some cases also be coated or otherwise treated to make the droplet operations surface hydrophobic. When the substrate includes a PCB, the following materials are examples of suitable materials: MITSUI™ BN-300 (available from MITSUI Chemicals America, Inc., San Jose Calif.); ARLON™ 11N (available from Arlon, Inc, Santa Ana, Calif.).; NELCO® N4000-6 and N5000-30/32 (available from Park Electrochemical Corp., Melville, N.Y.); ISOLA™ FR406 (available from Isola Group, Chandler, Ariz.), especially IS620; fluoropolymer family (suitable for fluorescence detection since it has low background fluorescence); polyimide family; polyester; polyethylene naphthalate; polycarbonate; polyetheretherketone; liquid crystal polymer; cyclo-olefin copolymer (COC); cyclo-olefin polymer (COP); aramid; THERMOUNT® nonwoven aramid reinforcement (available from DuPont, Wilmington, Del.); NOMEX® brand fiber (available from DuPont, Wilmington, Del.); and paper. Various materials are also suitable for use as the dielectric component of the substrate. Examples include: vapor deposited dielectric, such as PARYLENE™ C (especially on glass) and PARYLENE™ N (available from Parylene Coating Services, Inc., Katy, Tex.); TEFLON® AF coatings; cytop; soldermasks, such as liquid photoimageable soldermasks (e.g., on PCB) like TAIYO™ PSR4000 series, TAIYO™ PSR and AUS series (available from Taiyo America, Inc. Carson City, Nev.) (good thermal characteristics for applications involving thermal control), and PROBIMER™ 8165 (good thermal characteristics for applications involving thermal control (available from Huntsman Advanced Materials Americas Inc., Los Angeles, Calif.); dry film soldermask, such as those in the VACREL® dry film soldermask line (available from DuPont, Wilmington, Del.); film dielectrics, such as polyimide film (e.g., KAPTON® polyimide film, available from DuPont, Wilmington, Del.), polyethylene, and fluoropolymers (e.g., FEP), polytetrafluoroethylene; polyester; polyethylene naphthalate; cyclo-olefin copolymer (COC); cyclo-olefin polymer (COP); any other PCB substrate material listed above; black matrix resin; and polypropylene. Droplet transport voltage and frequency may be selected for performance with reagents used in specific assay protocols. Design parameters may be varied, e.g., number and placement of on-actuator reservoirs, number of independent electrode connections, size (volume) of different reservoirs, placement of magnets/bead washing zones, electrode size, inter-electrode pitch, and gap height (between top and bottom substrates) may be varied for use with specific reagents, protocols, droplet volumes, etc. In some cases, a substrate of the invention may derivatized with low surface-energy materials or chemistries, e.g., using deposition or in situ synthesis using poly- or per-fluorinated compounds in solution or polymerizable monomers. Examples include TEFLON® AF coatings and FLUOROPEL® coatings for dip or spray coating, and other fluorinated monomers for plasma-enhanced chemical vapor deposition (PECVD). Additionally, in some cases, some portion or all of the droplet operations surface may be coated with a substance for reducing background noise, such as background fluorescence from a PCB substrate. For example, the noise-reducing coating may include a black matrix resin, such as the black matrix resins available from Toray industries, Inc., Japan. Electrodes of a droplet actuator are typically controlled by a controller or a processor, which is itself provided as part of a system, which may include processing functions as well as data and software storage and input and output capabilities. Reagents may be provided on the droplet actuator in the droplet operations gap or in a reservoir fluidly coupled to the droplet operations gap. The reagents may be in liquid form, e.g., droplets, or they may be provided in a reconstitutable form in the droplet operations gap or in a reservoir fluidly coupled to the droplet operations gap. Reconstitutable reagents may typically be combined with liquids for reconstitution. An example of reconstitutable reagents suitable for use with the invention includes those described in Meathrel, et al., U.S. Pat. No. 7,727,466, entitled “Disintegratable films for diagnostic devices,” granted on Jun. 1, 2010.

“Droplet operation” means any manipulation of a droplet on a droplet actuator. A droplet operation may, for example, include: loading a droplet into the droplet actuator; dispensing one or more droplets from a source droplet; splitting, separating or dividing a droplet into two or more droplets; transporting a droplet from one location to another in any direction; merging or combining two or more droplets into a single droplet; diluting a droplet; mixing a droplet; agitating a droplet; deforming a droplet; retaining a droplet in position; incubating a droplet; heating a droplet; vaporizing a droplet; cooling a droplet; disposing of a droplet; transporting a droplet out of a droplet actuator; other droplet operations described herein; and/or any combination of the foregoing. The terms “merge,” “merging,” “combine,” “combining” and the like are used to describe the creation of one droplet from two or more droplets. It should be understood that when such a term is used in reference to two or more droplets, any combination of droplet operations that are sufficient to result in the combination of the two or more droplets into one droplet may be used. For example, “merging droplet A with droplet B,” can be achieved by transporting droplet A into contact with a stationary droplet B, transporting droplet B into contact with a stationary droplet A, or transporting droplets A and B into contact with each other. The terms “splitting,” “separating” and “dividing” are not intended to imply any particular outcome with respect to volume of the resulting droplets (i.e., the volume of the resulting droplets can be the same or different) or number of resulting droplets (the number of resulting droplets may be 2, 3, 4, 5 or more). The term “mixing” refers to droplet operations which result in more homogenous distribution of one or more components within a droplet. Examples of “loading” droplet operations include microdialysis loading, pressure assisted loading, robotic loading, passive loading, and pipette loading. Droplet operations may be electrode-mediated. In some cases, droplet operations are further facilitated by the use of hydrophilic and/or hydrophobic regions on surfaces and/or by physical obstacles. For examples of droplet operations, see the patents and patent applications cited above under the definition of “droplet actuator.”Impedance or capacitance sensing or imaging techniques may sometimes be used to determine or confirm the outcome of a droplet operation. Examples of such techniques are described in Sturmer et al., International Patent Pub. No. WO/2008/101194, entitled “Capacitance Detection in a Droplet Actuator,” published on Aug. 21, 2008, the entire disclosure of which is incorporated herein by reference. Generally speaking, the sensing or imaging techniques may be used to confirm the presence or absence of a droplet at a specific electrode. For example, the presence of a dispensed droplet at the destination electrode following a droplet dispensing operation confirms that the droplet dispensing operation was effective. Similarly, the presence of a droplet at a detection spot at an appropriate step in an assay protocol may confirm that a previous set of droplet operations has successfully produced a droplet for detection. Droplet transport time can be quite fast. For example, in various embodiments, transport of a droplet from one electrode to the next may exceed about 1 sec, or about 0.1 sec, or about 0.01 sec, or about 0.001 sec. In one embodiment, the electrode is operated in AC mode but is switched to DC mode for imaging. It is helpful for conducting droplet operations for the footprint area of droplet to be similar to electrowetting area; in other words, 1x-, 2x- 3x-droplets are usefully controlled operated using 1, 2, and 3 electrodes, respectively. If the droplet footprint is greater than the number of electrodes available for conducting a droplet operation at a given time, the difference between the droplet size and the number of electrodes should typically not be greater than 1; in other words, a 2x droplet is usefully controlled using 1 electrode and a 3x droplet is usefully controlled using 2 electrodes. When droplets include beads, it is useful for droplet size to be equal to the number of electrodes controlling the droplet, e.g., transporting the droplet.

“Filler fluid” means a fluid associated with a droplet operations substrate of a droplet actuator, which fluid is sufficiently immiscible with a droplet phase to render the droplet phase subject to electrode-mediated droplet operations. For example, the gap of a droplet actuator is typically filled with a filler fluid. The filler fluid may, for example, be a low-viscosity oil, such as silicone oil or hexadecane filler fluid. The filler fluid may fill the entire gap of the droplet actuator or may coat one or more surfaces of the droplet actuator. Filler fluids may be conductive or non-conductive. Filler fluids may, for example, be doped with surfactants or other additives. For example, additives may be selected to improve droplet operations and/or reduce loss of reagent or target substances from droplets, formation of microdroplets, cross contamination between droplets, contamination of droplet actuator surfaces, degradation of droplet actuator materials, etc. Composition of the filler fluid, including surfactant doping, may be selected for performance with reagents used in the specific assay protocols and effective interaction or non-interaction with droplet actuator materials. Examples of filler fluids and filler fluid formulations suitable for use with the invention are provided in Srinivasan et al, International Patent Pub. Nos. WO/2010/027894, entitled “Droplet Actuators, Modified Fluids and Methods,” published on Mar. 11, 2010, and WO/2009/021173, entitled “Use of Additives for Enhancing Droplet Operations,” published on Feb. 12, 2009; Sista et al., International Patent Pub. No. WO/2008/098236, entitled “Droplet Actuator Devices and Methods Employing Magnetic Beads,” published on Aug. 14, 2008; and Monroe et al., U.S. Patent Publication No. 20080283414, entitled “Electrowetting Devices,” filed on May 17, 2007; the entire disclosures of which are incorporated herein by reference, as well as the other patents and patent applications cited herein.

“Immobilize” with respect to magnetically responsive beads, means that the beads are substantially restrained in position in a droplet or in filler fluid on a droplet actuator. For example, in one embodiment, immobilized beads are sufficiently restrained in position in a droplet to permit execution of a droplet splitting operation, yielding one droplet with substantially all of the beads and one droplet substantially lacking in the beads.

“Magnetically responsive” means responsive to a magnetic field. “Magnetically responsive beads” include or are composed of magnetically responsive materials. Examples of magnetically responsive materials include paramagnetic materials, ferromagnetic materials, ferrimagnetic materials, and metamagnetic materials. Examples of suitable paramagnetic materials include iron, nickel, and cobalt, as well as metal oxides, such as Fe₃O₄, BaFe₁₂O₁₉, CoO, NiO, Mn₂O₃, Cr₂O₃, and CoMnP.

“Reservoir” means an enclosure or partial enclosure configured for holding, storing, or supplying liquid. A droplet actuator system of the invention may include on-cartridge reservoirs and/or off-cartridge reservoirs. On-cartridge reservoirs may be (1) on-actuator reservoirs, which are reservoirs in the droplet operations gap or on the droplet operations surface; (2) off-actuator reservoirs, which are reservoirs on the droplet actuator cartridge, but outside the droplet operations gap, and not in contact with the droplet operations surface; or (3) hybrid reservoirs which have on-actuator regions and off-actuator regions. An example of an off-actuator reservoir is a reservoir in the top substrate. An off-actuator reservoir is typically in fluid communication with an opening or fluid path arranged for flowing liquid from the off-actuator reservoir into the droplet operations gap, such as into an on-actuator reservoir. An off-cartridge reservoir may be a reservoir that is not part of the droplet actuator cartridge at all, but which flows liquid to some portion of the droplet actuator cartridge. For example, an off-cartridge reservoir may be part of a system or docking station to which the droplet actuator cartridge is coupled during operation. Similarly, an off-cartridge reservoir may be a reagent storage container or syringe which is used to force fluid into an on-cartridge reservoir or into a droplet operations gap. A system using an off-cartridge reservoir will typically include a fluid passage means whereby liquid may be transferred from the off-cartridge reservoir into an on-cartridge reservoir or into a droplet operations gap.

“Transporting into the magnetic field of a magnet,” “transporting towards a magnet,” and the like, as used herein to refer to droplets and/or magnetically responsive beads within droplets, is intended to refer to transporting into a region of a magnetic field capable of substantially attracting magnetically responsive beads in the droplet. Similarly, “transporting away from a magnet or magnetic field,” “transporting out of the magnetic field of a magnet,” and the like, as used herein to refer to droplets and/or magnetically responsive beads within droplets, is intended to refer to transporting away from a region of a magnetic field capable of substantially attracting magnetically responsive beads in the droplet, whether or not the droplet or magnetically responsive beads is completely removed from the magnetic field. It will be appreciated that in any of such cases described herein, the droplet may be transported towards or away from the desired region of the magnetic field, and/or the desired region of the magnetic field may be moved towards or away from the droplet. Reference to an electrode, a droplet, or magnetically responsive beads being “within” or “in” a magnetic field, or the like, is intended to describe a situation in which the electrode is situated in a manner which permits the electrode to transport a droplet into and/or away from a desired region of a magnetic field, or the droplet or magnetically responsive beads is/are situated in a desired region of the magnetic field, in each case where the magnetic field in the desired region is capable of substantially attracting any magnetically responsive beads in the droplet. Similarly, reference to an electrode, a droplet, or magnetically responsive beads being “outside of” or “away from” a magnetic field, and the like, is intended to describe a situation in which the electrode is situated in a manner which permits the electrode to transport a droplet away from a certain region of a magnetic field, or the droplet or magnetically responsive beads is/are situated away from a certain region of the magnetic field, in each case where the magnetic field in such region is not capable of substantially attracting any magnetically responsive beads in the droplet or in which any remaining attraction does not eliminate the effectiveness of droplet operations conducted in the region. In various aspects of the invention, a system, a droplet actuator, or another component of a system may include a magnet, such as one or more permanent magnets (e.g., a single cylindrical or bar magnet or an array of such magnets, such as a Halbach array) or an electromagnet or array of electromagnets, to form a magnetic field for interacting with magnetically responsive beads or other components on chip. Such interactions may, for example, include substantially immobilizing or restraining movement or flow of magnetically responsive beads during storage or in a droplet during a droplet operation or pulling magnetically responsive beads out of a droplet.

“Washing” with respect to washing a bead means reducing the amount and/or concentration of one or more substances in contact with the bead or exposed to the bead from a droplet in contact with the bead. The reduction in the amount and/or concentration of the substance may be partial, substantially complete, or even complete. The substance may be any of a wide variety of substances; examples include target substances for further analysis, and unwanted substances, such as components of a sample, contaminants, and/or excess reagent. In some embodiments, a washing operation begins with a starting droplet in contact with a magnetically responsive bead, where the droplet includes an initial amount and initial concentration of a substance. The washing operation may proceed using a variety of droplet operations. The washing operation may yield a droplet including the magnetically responsive bead, where the droplet has a total amount and/or concentration of the substance which is less than the initial amount and/or concentration of the substance. Examples of suitable washing techniques are described in Pamula et al., U.S. Pat. No. 7,439,014, entitled “Droplet-Based Surface Modification and Washing,” granted on Oct. 21, 2008, the entire disclosure of which is incorporated herein by reference.

The terms “top,” “bottom,” “over,” “under,” and “on” are used throughout the description with reference to the relative positions of components of the droplet actuator, such as relative positions of top and bottom substrates of the droplet actuator. It will be appreciated that the droplet actuator is functional regardless of its orientation in space.

When a liquid in any form (e.g., a droplet or a continuous body, whether moving or stationary) is described as being “on”, “at”, or “over” an electrode, array, matrix or surface, such liquid could be either in direct contact with the electrode/array/matrix/surface, or could be in contact with one or more layers or films that are interposed between the liquid and the electrode/array/matrix/surface.

When a droplet is described as being “on” or “loaded on” a droplet actuator, it should be understood that the droplet is arranged on the droplet actuator in a manner which facilitates using the droplet actuator to conduct one or more droplet operations on the droplet, the droplet is arranged on the droplet actuator in a manner which facilitates sensing of a property of or a signal from the droplet, and/or the droplet has been subjected to a droplet operation on the droplet actuator.

### 6 BRIEF DESCRIPTION OF THE DRAWINGS

FIG. 1 illustrates a cross-sectional view of an example of a portion of a droplet actuator in contact with a sonication mechanism for promoting cell lysis;

FIG. 2 illustrates a cross-sectional view of an example of a sample reservoir assembly that incorporates a sonication mechanism for promoting cell lysis;

FIG. 3 illustrates another cross-sectional view of a portion of the droplet actuator of FIG. 1 in contact with a sonication mechanism for promoting cell lysis;

FIGS. 4A, 4B, and 4C illustrate an example of a process of using a sonication mechanism by which different power levels may be delivered to the sample in a droplet actuator;

FIG. 5 illustrates another cross-sectional view of a portion of the droplet actuator of FIG. 1 in contact with a sonication mechanism for promoting cell lysis;

FIG. 6 illustrates another cross-sectional view of a portion of the droplet actuator of FIG. 1 in contact with a sonication mechanism for promoting cell lysis;

FIG. 7 illustrates another cross-sectional view of a portion of the droplet actuator of FIG. 1 in contact with a sonication mechanism for promoting cell lysis;

FIG. 8 illustrates another cross-sectional view of a portion of the droplet actuator of FIG. 1 in contact with a sonication mechanism for promoting cell lysis;

FIGS. 9A and 9B illustrate a top and side view, respectively, of an on-chip sonication mechanism, which is an example of a sonication mechanism that is integrated into a droplet actuator for promoting cell lysis;

FIG. 10 illustrates a cross-sectional view of an on-chip sonication mechanism, which is yet another example of a sonication mechanism that is integrated into a droplet actuator for promoting cell lysis;

FIG. 11A illustrates another cross-sectional view of a portion of the droplet actuator of FIG. 1 in contact with a sonication mechanism for promoting cell lysis;

FIG. 11B illustrates a top view of the example sonication mechanism of FIG. 11A;

FIG. 12A illustrates yet another cross-sectional view of a portion of the droplet actuator of FIG. 1 and shows an example of a heating mechanism coupled thereto for promoting cell lysis;

FIG. 12B illustrates a top view of the example heating mechanism of FIG. 12A;

FIG. 13 illustrates yet another cross-sectional view of a portion of the droplet actuator of FIG. 1 and shows an example of using a laser as the heat source for promoting cell lysis;

FIG. 14 illustrates yet another cross-sectional view of a portion of the droplet actuator of FIG. 1 and shows an example of incorporating the combination of a sonication mechanism and a heating mechanism for promoting cell lysis;

FIG. 15 illustrates yet another cross-sectional view of a portion of the droplet actuator of FIG. 1 shows another example of incorporating the combination of a sonication mechanism and a heating mechanism for promoting cell lysis;

FIG. 16 illustrates yet another cross-sectional view of a portion of the droplet actuator of FIG. 1 and shows an example of using mechanical shearing for promoting cell lysis;

FIG. 17 illustrates yet another cross-sectional view of a portion of the droplet actuator of FIG. 1 and shows another example of using mechanical shearing for promoting cell lysis;

FIGS. 18A, 18B, and 18C illustrate certain views of yet other examples of mechanisms for causing mechanical shearing in a droplet actuator;

FIG. 19 illustrates another cross-sectional view of a portion of the droplet actuator of FIG. 1 and shows another example of using mechanical shearing for promoting cell lysis;

FIG. 20A illustrates yet another cross-sectional view of a portion of the droplet actuator of FIG. 1 and shows another example of using mechanical shearing for promoting cell lysis;

FIG. 20B illustrates a top view of one example of a grinding mechanism that is suitable for causing cell disruption;

FIG. 20C illustrates a top view of another example of a grinding mechanism that is suitable for causing cell disruption;

FIG. 21 illustrates yet another cross-sectional view of a portion of the droplet actuator of FIG. 1 and shows an example of using electrically-induced bead beating for promoting cell lysis;

FIG. 22 illustrates yet another cross-sectional view of a portion of the droplet actuator of FIG. 1 and shows an example of using magnetically-induced bead beating for promoting cell lysis;

FIG. 23 illustrates yet another cross-sectional view of a portion of the droplet actuator of FIG. 1 and shows an example of using electrically-induced bead beating for promoting cell lysis;

FIG. 24 illustrates yet another cross-sectional view of a portion of the droplet actuator of FIG. 1 and shows an example of using magnetically-induced bead beating for promoting cell lysis;

FIG. 25 illustrates yet another cross-sectional view of a portion of the droplet actuator of FIG. 1 and shows another example of using electrically-induced bead beating for promoting cell lysis.

FIG. 26 illustrates yet another cross-sectional view of a portion of the droplet actuator of FIG. 1 and shows an example of using laser-assisted bead beating for promoting cell lysis;

FIG. 27 illustrates yet another cross-sectional view of a portion of the droplet actuator of FIG. 1 and shows an example of features incorporated therein that promote ultrasonic cavitation and, thereby, promote cell lysis;

FIG. 28 illustrates a cross-sectional view of an example of a portion of the droplet actuator of FIG. 1 that includes a barrier for retaining microemulsion droplets that may result from sonication and a process of collecting the microemulsion droplets;

FIG. 29 illustrates yet another top view of a portion of the droplet actuator of FIG. 1 and shows an example of using electric fields for promoting cell lysis;

FIG. 30 illustrates yet another top view of a portion of the droplet actuator of FIG. 1 and shows another example of using electric fields for promoting cell lysis;

FIG. 31 illustrates yet another top view of a portion of the droplet actuator of FIG. 1 and shows another example of using electric fields for promoting cell lysis;

FIG. 32 illustrates yet another cross-sectional view of a portion of the droplet actuator of FIG. 1 and shows another example of using electric fields for promoting cell lysis;

FIG. 33 illustrates yet another cross-sectional view of a portion of the droplet actuator of FIG. 1 and shows another example of using electric fields for promoting cell lysis;

FIG. 34 illustrates yet another cross-sectional view of a portion of the droplet actuator of FIG. 1 and shows another example of using electric fields for promoting cell lysis;

FIG. 35 illustrates yet another cross-sectional view of a portion of the droplet actuator of FIG. 1 and shows another example of using electric fields for promoting cell lysis;

FIG. 36 illustrates yet another cross-sectional view of a portion of the droplet actuator of FIG. 1 and shows another example of using electric fields for promoting cell lysis;

FIGS. 37A, 37B, and 37C illustrate yet other cross-sectional and top views of a portion of the droplet actuator of FIG. 1 and show another example of using electric fields for promoting cell lysis;

FIG. 38 illustrates yet another cross-sectional view of a portion of the droplet actuator of FIG. 1 and shows an example of using thermal cycling for promoting cell lysis;

FIG. 39 illustrates yet another cross-sectional view of a portion of the droplet actuator of FIG. 1 and shows an example of using thermal cycling for promoting cell lysis; and

FIG. 40 illustrates a cross-sectional view of a dounce homogenizer that may be used for promoting cell lysis in a cell-containing sample fluid by mechanical shearing.

FIG. 41 illustrates a droplet actuator system in accordance with an embodiment of the invention.

### 7 DETAILED DESCRIPTION OF THE INVENTION

The present invention relates to systems, devices and methods for promoting disruption of materials, such as tissues, cells and spores in a droplet actuator system, cartridge or chip. In certain embodiments, sonication mechanisms are used with droplet actuators to promote disruption of materials associated with the droplet actuator, such as materials in a droplet on a substrate of a droplet actuator. In one example, a droplet actuator system may incorporate an ultrasonic probe that is in contact with a wall of a sample reservoir of the droplet actuator, such as a sample reservoir mounted on a top substrate.

In another example, a droplet actuator system may incorporate a sonic probe that is in contact with the top substrate. In yet another example, a droplet actuator system may incorporate a sonic probe that is in contact with the bottom substrate. In still another example, a droplet actuator may incorporate certain built in structures for creating ultrasonic vibration.

In other embodiments, heating mechanisms or the combination of both heating mechanisms and sonication mechanisms may be used to promote cell lysis in droplet actuators. In yet other embodiments, mechanical shearing mechanisms may be used to promote disruption of materials in droplet actuator cartridges or chips. In yet other embodiments, bead beating mechanisms may be used to promote disruption of materials in droplet actuator cartridges or chips. In yet other embodiments, certain physical features may be incorporated into a droplet actuator for promoting ultrasonic cavitation and, thereby, promoting disruption of materials in droplet actuator cartridges or chips. In yet other embodiments, electric fields may be used to promote disruption of materials in droplet actuator cartridges or chips. In still other embodiments, thermal cycling may be used to promote disruption of materials in droplet actuator cartridges or chips.

In the various examples that follow, cell lysis is used as an exemplary embodiment; however, it will be appreciated that the invention is useful for disrupting any materials, such as lysing cells or spores, breaking apart tissues, breaking apart particles, etc.

### 7.1 Cell Lysis by Sonication

Lysis refers to the breaking down of a cell (or cell disruption), which may occur by any mechanism that compromise the cell's integrity. Cell lysis methods through cell rupture can be classified into mechanical methods and non-mechanical methods. Sonication is one example of a mechanical cell lysis method. Sonication applies ultrasound (typically 20-50 kilohertz (kHz)) to a cell-containing sample. In principle, the high-frequency is generated electronically and the mechanical energy may be transmitted to the sample via, for example, a probe that oscillates with high frequency. When ultrasonic energy is transmitted to the cell-containing sample, the high-frequency oscillation causes a localized low pressure region that results in cavitation and impaction, ultimately breaking open the cells. Disclosed herein are novel systems, structures, and/or methods of using sonication in droplet actuators for promoting cell lysis in sample droplets and/or in any volumes of cell-containing sample fluid. In other embodiments, sonication may be used to agitate particles or break molecular interactions.

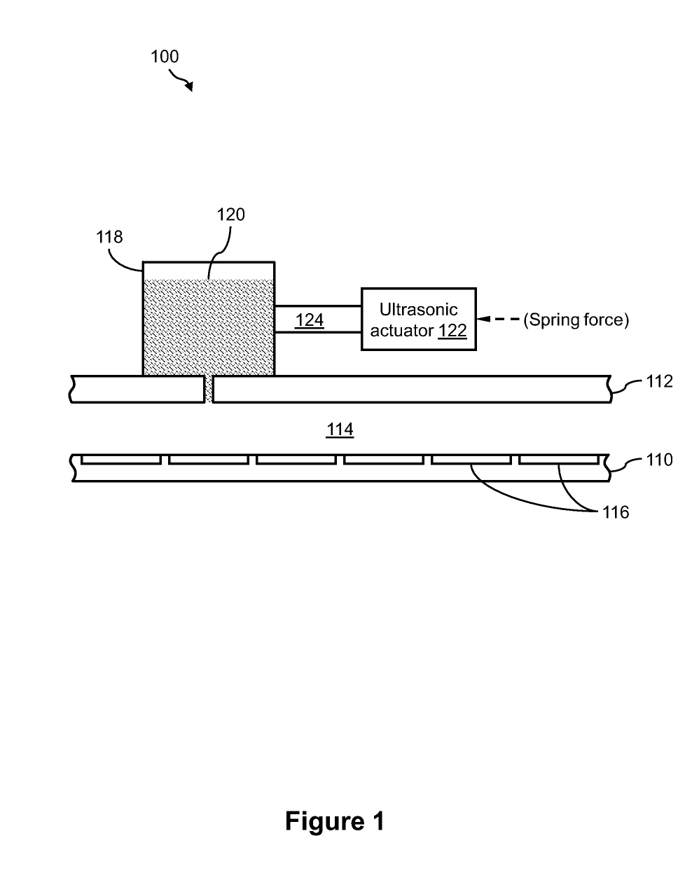

FIG. 1 illustrates a cross-sectional view of an example of a portion of a droplet actuator 100 in association with a sonication mechanism for promoting cell lysis. Droplet actuator 100 may include a bottom substrate 110 that is separated from a top substrate 112 by a gap 114. A spacer (not shown) may be used to determine the size of gap 114. Bottom substrate 110 may be formed, for example, of a printed circuit board (PCB). Top substrate 112 may be formed, for example, of glass, plastic, PCB, and/or indium tin oxide (ITO). Bottom substrate 110 may include an arrangement of droplet operations electrodes 116 (e.g., electrowetting electrodes). Droplet operations are conducted atop droplet operations electrodes 116 on a droplet operations surface. In an another embodiment droplet operations electrodes 116 may be arranged on one or both of bottom substrate 110 and top substrate 112.

Associated with top substrate 112 is a sample reservoir 118 for holding a quantity of sample fluid 120 that contains cells to be lysed. In this embodiment, the sonication mechanism or device for promoting cell lysis is an ultrasonic actuator 122. In one example, ultrasonic actuator 122 may be a commercially available 40 kHz sonic probe. A tip 124 of ultrasonic actuator 122 is in association with a side of sample reservoir 118. In one example tip 124 may be pressed the side of sample reservoir 118 by spring force. Sample reservoir 118 may be formed, for example, of injection-molded plastic. The walls of sample reservoir 118, or at least the wall portion associated with tip 124 of ultrasonic actuator 122, may be suitability thin to ensure efficient transfer of ultrasonic energy from ultrasonic actuator 122 to sample fluid 120. In one example, the walls of sample reservoir 118 may be about 0.5 millimeters (mm) or less in thickness.

In operation, tip 124 of ultrasonic actuator 122 is in associations with the side of sample reservoir 118, e.g., ultrasonic actuator 122 may be pressed by spring force against the side of sample reservoir 118. Ultrasonic actuator 122 is activated and the ultrasonic energy from tip 124 is transferred through the wall of sample reservoir 118 and to sample fluid 120 that contains cells to be lysed. Due to the ultrasonic energy from ultrasonic actuator 122, a cell lysis process occurs in sample fluid 120. A fluid containing the contents of lysed cells is called a “lysate.” As a result of the sonication, sample fluid 120 is now lysate and may be delivered into gap 114 of droplet actuator 100 for processing.

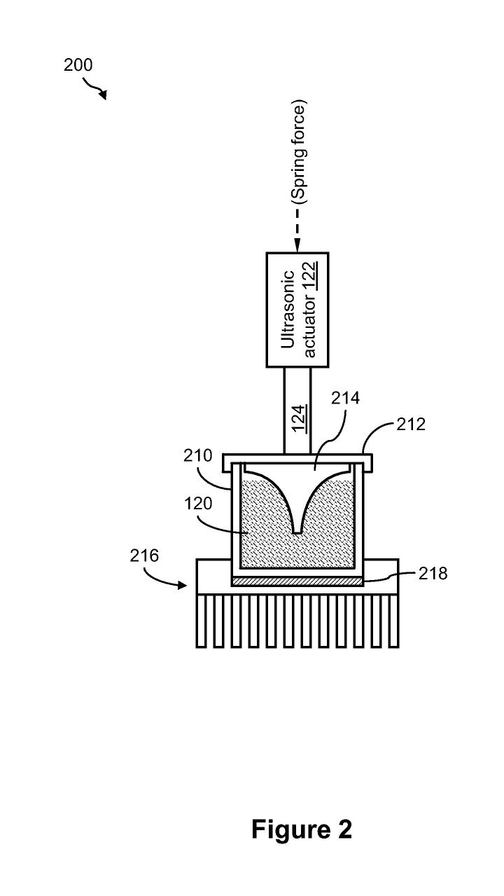

FIG. 2 illustrates a cross-sectional view of an example of a sample reservoir assembly 200 that incorporates a sonication mechanism for promoting cell lysis. Sample reservoir assembly 200 is suitable for use with any droplet actuator, such as droplet actuator 100 of FIG. 1. Sample reservoir assembly 200 includes a sample reservoir 210 for holding a quantity of the cell-containing sample fluid 120 that is described in FIG. 1. Sample reservoir 210 may be formed, for example, of injection-molded plastic. A cap 212, also which may be formed of injection-molded plastic, is positioned atop sample reservoir 210. Further, an ultrasonic horn 214 is integrated into cap 212 in a manner whereby ultrasonic horn 214 is submerged, or partially submerged, in sample fluid 120 when installed. Generally, an ultrasonic horn is a device used to pass ultrasound into a liquid medium. When in use, a sonication mechanism, such as ultrasonic actuator 122, is preferably in close association with cap 212. In one example, ultrasonic actuator 122 may be pressed by spring force, or other suitable means, against cap 212. In this way, the ultrasonic energy is transferred to ultrasonic horn 214 and then to the cell-containing sample fluid 120 for causing cell lysis to occur therein. In this configuration, the integrated cap 212 and ultrasonic horn 214 component may be a disposable element of sample reservoir assembly 200. In this scenario, an emersion type of sonication may occur without the risk of contaminating ultrasonic actuator 122, which may be shared across multiple samples.

Heat may be generated during the sonication process. Therefore, sample reservoir assembly 200 may include certain heat removal mechanisms that are in thermal contact with sample reservoir 210. For example, sample reservoir assembly 200 may be mounted atop an air cooled heat sink 216 and/or any other cooling mechanism 218, such as, but not limited to, a Peltier cooler, which is a thermoelectric cooling device.

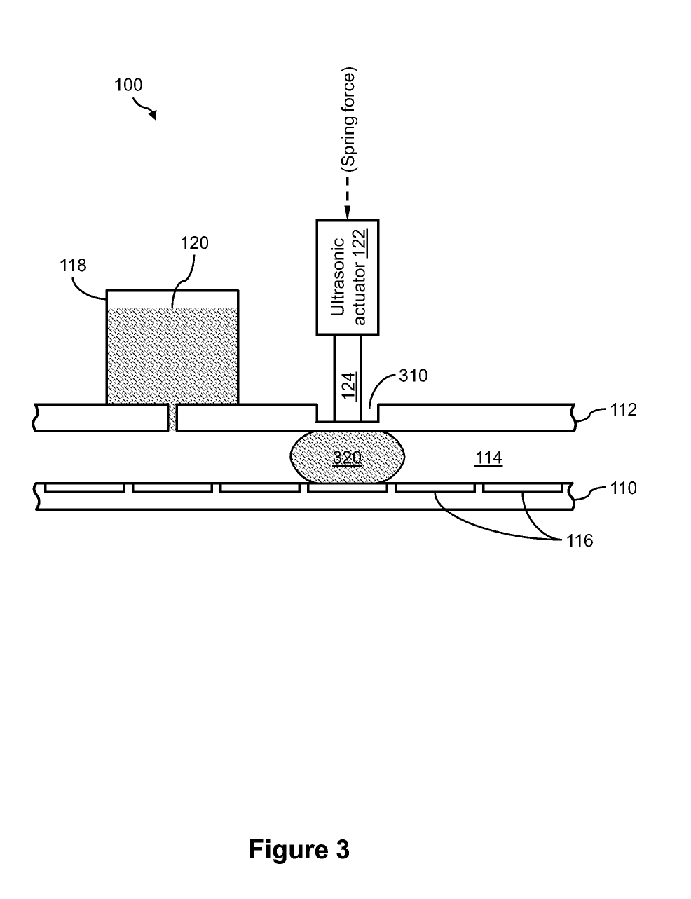

FIG. 3 illustrates another cross-sectional view of a portion of droplet actuator 100 of FIG. 1 in association with a sonication mechanism for promoting cell lysis. In this example, a sonication mechanism is associated with a substrate, e.g., top substrate 112, of droplet actuator 100. For example, top substrate 112 of droplet actuator 100 may include a recessed region 310 for accepting tip 124 of ultrasonic actuator 122. Recessed region 310 substantially aligns with a certain droplet operations electrode 116. The presence of recessed region 310 in top substrate 112 creates a thin region of top substrate 112 through which ultrasonic energy may pass.

In operation, a cell-containing sample droplet 320 may be transported via droplet operations to the droplet operations electrode 116 that is substantially aligned with recessed region 310. Tip 124 of ultrasonic actuator 122, which is positioned at recessed region 310, is in association with the thin region of top substrate 112. In one example, tip 124 of ultrasonic actuator 122, is pressed by spring force against the thin region of top substrate 112. Ultrasonic actuator 122 is activated and the ultrasonic energy from tip 124 is transferred through the thin region of the substrate, e.g., top substrate 112, and to sample droplet 320 that contains cells to be lysed. Due to the ultrasonic energy from ultrasonic actuator 122, a cell lysis process occurs in sample droplet 320. As a result of the sonication, sample droplet 320 is now lysate, which may be further processed in gap 114 of droplet actuator 100.

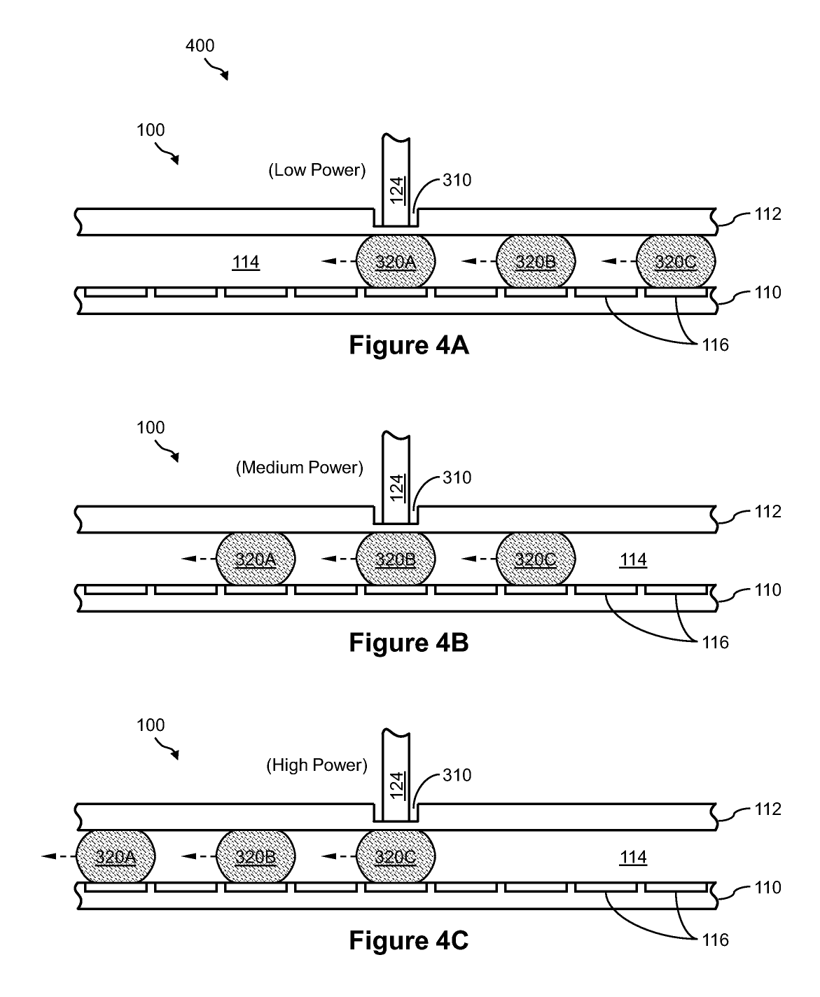

FIGS. 4A, 4B, and 4C illustrate an example of a process 400 of using a sonication mechanism by which different power levels may be delivered to the sample in a droplet actuator. In this example, droplet actuator 100 is used that includes a sonication mechanism in association with a substrate of droplet actuator 100, e.g., top substrate 112, as described with respect to FIG. 3. By way of example, FIGS. 4A, 4B, and 4C show three cell-containing sample droplets 320A-C present in gap 114 of droplet actuator 100.

Sample droplets 320 may contain different types of cells, requiring different ultrasonic energy levels, respectively, for causing cell lysis. For example, a sample droplet 320A may be a viral sample droplet wherein cell lysis may occur using a low level of ultrasonic energy. A sample droplet 320B may be a bacterial sample droplet wherein cell lysis may occur using a higher level of ultrasonic energy. A sample droplet 320C may be a fungal sample droplet wherein cell lysis may occur using a yet higher level of ultrasonic energy.

Referring to FIG. 4A, sample droplet 320A may be transported via droplet operations to the droplet operations electrode 116 that is substantially aligned with recessed region 310 and ultrasonic actuator 122. Ultrasonic actuator 122 is activated at a certain low energy level that is suitable for causing cell lysis to occur in sample droplet 320A, which may be a virus cell-containing sample droplet.

Referring to FIG. 4B, sample droplet 320A is transported via droplet operations away from ultrasonic actuator 122, while sample droplet 320B is transported to the droplet operations electrode 116 that is substantially aligned with recessed region 310 and ultrasonic actuator 122. Ultrasonic actuator 122 is activated at a certain higher energy level that is suitable for causing cell lysis to occur in sample droplet 320B, which, in one example, may be a bacteria cell-containing sample droplet.

Referring to FIG. 4C, sample droplet 320A and sample droplet 320B are transported via droplet operations away from ultrasonic actuator 122, while sample droplet 320C is transported to the droplet operations electrode 116 that is substantially aligned with recessed region 310 and ultrasonic actuator 122. Ultrasonic actuator 122 is activated at a yet higher energy level that is suitable for causing cell lysis to occur in sample droplet 320C, which, in one example, may be a fungus cell-containing sample droplet.

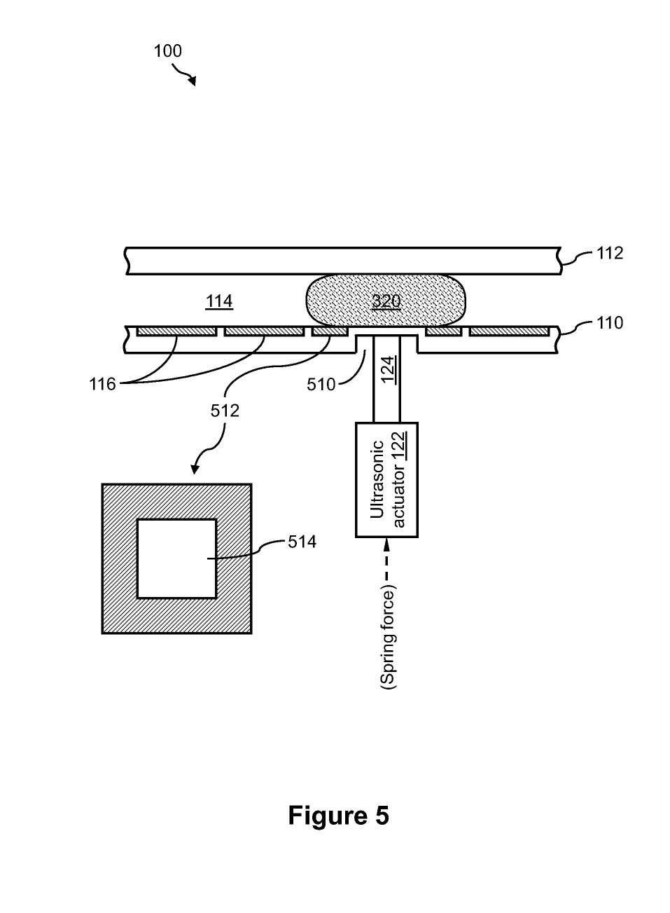

FIG. 5 illustrates another cross-sectional view of a portion of droplet actuator 100 of FIG. 1 in association with a sonication mechanism for promoting cell lysis. In this example, a sonication mechanism is in association with a substrate, e.g., bottom substrate 110, of a droplet actuator. For example, bottom substrate 110 of droplet actuator 100 may include a recessed region 510 for accepting tip 124 of ultrasonic actuator 122. The presence of recessed region 510 in bottom substrate 110 creates a thin region of bottom substrate 110 through which ultrasonic energy may pass. Further, a specially shaped droplet operations electrode 512 that includes a clearance region 514 is provided. Droplet operations electrode 512 substantially aligns with recessed region 510. In one example, the sonication mechanism is in contact with bottom substrate 110 of droplet actuator 100.

In operation, a cell-containing sample droplet 320 may be transported via droplet operations to the droplet operations electrode 512 that is substantially aligned with recessed region 510, Tip 124 of the sonication device, i.e., an ultrasonic actuator 122, which is positioned at recessed region 510, is in association with the thin region of bottom substrate 110. In one example, tip 124 of ultrasonic actuator 122, positioned at recessed region 510, is pressed by spring force against the thin region of bottom substrate 110. Ultrasonic actuator 122 is activated and the ultrasonic energy from tip 124 is transferred through the thin region of bottom substrate 110 and to sample droplet 320 that contains cells to be lysed. Clearance region 514 is present in droplet operations electrode 512 to assist the ultrasonic energy to pass from ultrasonic actuator 122 to sample droplet 320.

Due to the ultrasonic energy from ultrasonic actuator 122, a cell lysis process occurs in sample droplet 320. As a result, sample droplet 320 is now lysate, which may be further processed in gap 114 of droplet actuator 100.

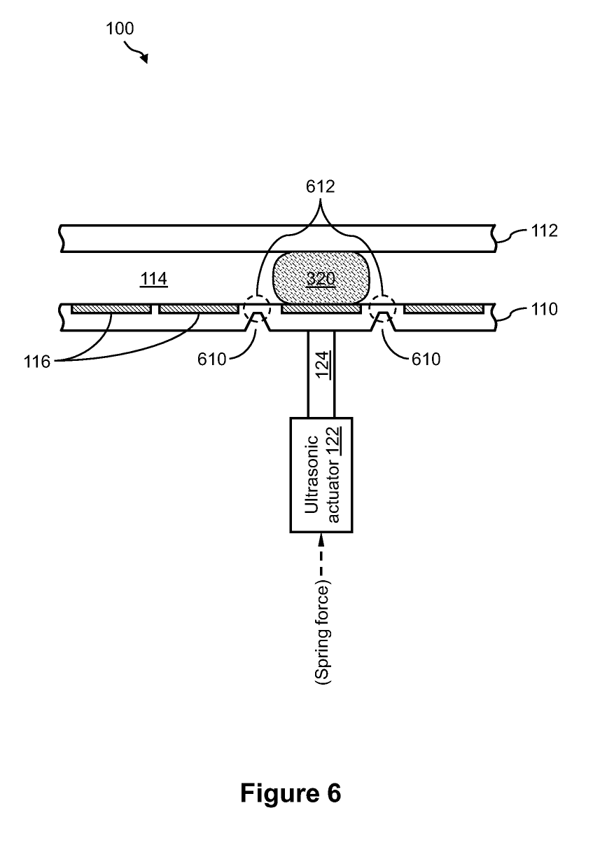

FIG. 6 illustrates another cross-sectional view of a portion of droplet actuator 100 of FIG. 1 in association with a sonication mechanism for promoting cell lysis. FIG. 6 shows another example of a sonication mechanism in association with a substrate, e.g., bottom substrate 110, of droplet actuator 100. For example, bottom substrate 110 of droplet actuator 100 may include a channel 610 that may be etched or routed into the PCB material, for example of bottom substrate 110. Channel 610 forms a ring around a certain droplet operations electrode 116. The presence of channel 610 in bottom substrate 110 also creates a thin ring region 612 around the certain droplet operations electrode 116. The presence of thin ring region 612 around a certain droplet operations electrode 116 allows the portion of bottom substrate 110 within the thin ring region 612 to have a certain amount of flexibility when subjected to sonication. Therefore, a sonication mechanism may be in association with this flexible portion of bottom substrate 110 for supplying ultrasonic energy to any cell-containing sample droplet 320 that is present at this location. For example, FIG. 6 shows ultrasonic actuator 122 in association with, e.g., pressed by spring force, the flexible portion of bottom substrate 110 within the thin ring region 612. In doing so, ultrasonic energy may be supplied to a cell-containing sample droplet 320.

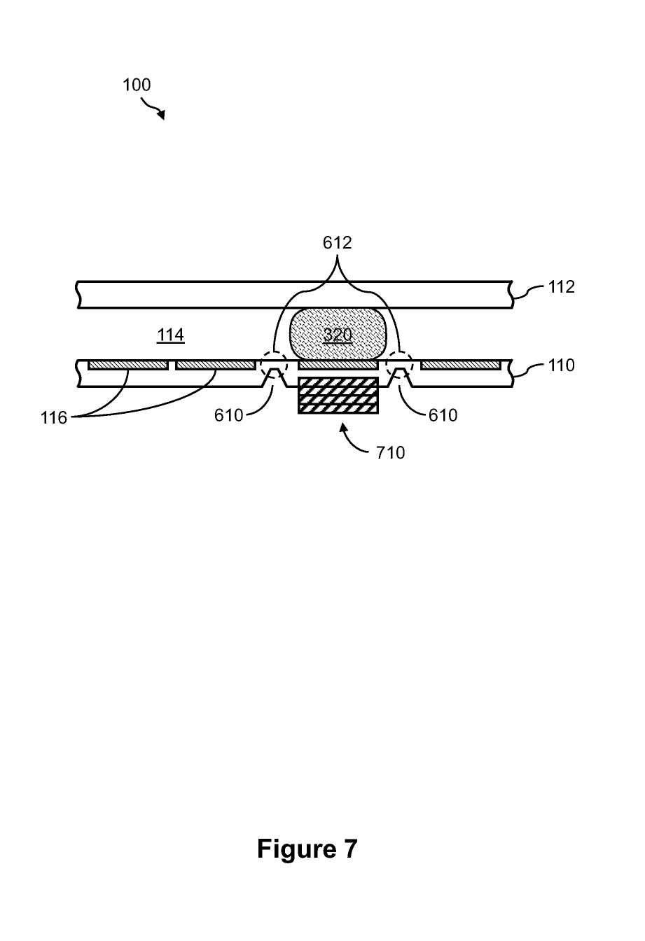

FIG. 7 illustrates another cross-sectional view of a portion of droplet actuator 100 of FIG. 1 in association with a sonication mechanism for promoting cell lysis. FIG. 7 shows yet another example of a sonication mechanism in association with a substrate, e.g., bottom substrate 110, of a droplet actuator 100. This sonication mechanism is substantially the same as the sonication mechanism of FIG. 6, except that ultrasonic actuator 122 is replaced with a built in sonication mechanism within the thin ring region 612 of the substrate, e.g., bottom substrate 110, which is the flexible portion of bottom substrate 110. For example, the built in sonication mechanism may be implemented as an on-chip piezoelectric stack 710. Piezoelectric stack 710 may be formed of a stack of any piezoelectric material, such as certain crystals and ceramics. One example of piezoelectric material is quartz (Si02). Referring again to FIGS. 6 and 7, the flexible portion of droplet actuator 100 for associating with the sonication mechanism is not limited to the bottom substrate only. The flexible portion may be incorporated in the bottom substrate, top substrate, and/or both substrates.

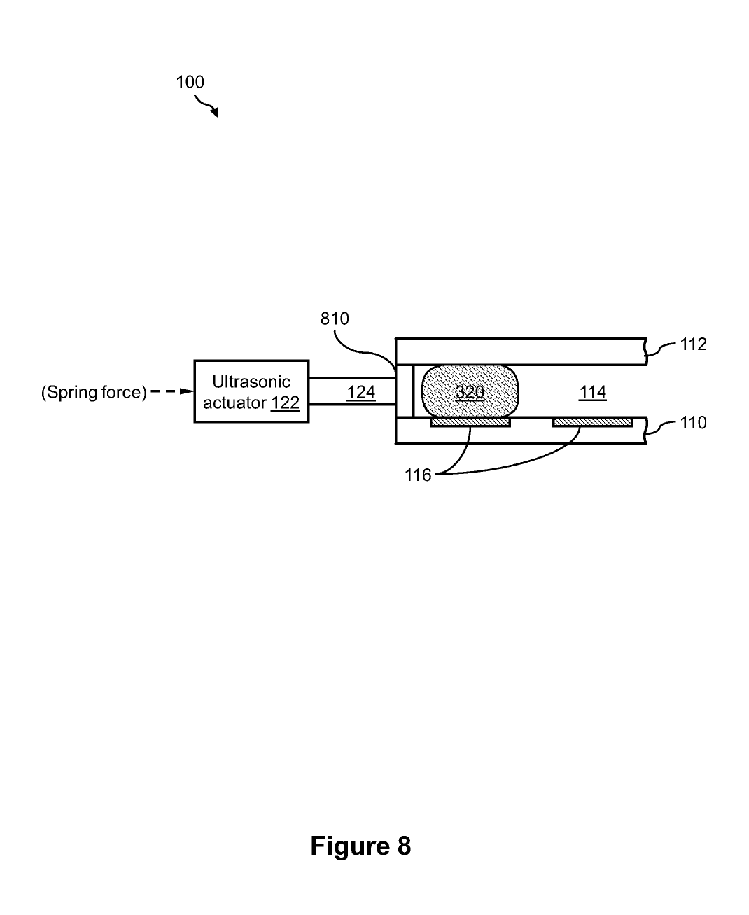

FIG. 8 illustrates another cross-sectional view of a portion of droplet actuator 100 of FIG. 1 in association with a sonication mechanism for promoting cell lysis. While FIGS. 1 through 7 show sonication mechanisms in association with, for example, bottom substrate 110 and/or top substrate 112 of a droplet actuator 100, FIG. 8 shows a sonication mechanism in association with the edge of a droplet actuator, which is in substantially the same plane as gap 114 of droplet actuator 100. For example, FIG. 8 shows tip 124 of ultrasonic actuator 122 in association with, e.g., pressed by spring force against, a spacer 810 at the edge of droplet actuator 100. Spacer 810 is between bottom substrate 110 and top substrate 112 and may determine the height of gap 114. In this way, ultrasonic energy may be delivered to the cell-containing sample droplet 320 in a direction that is substantially along the same plane as gap 114.

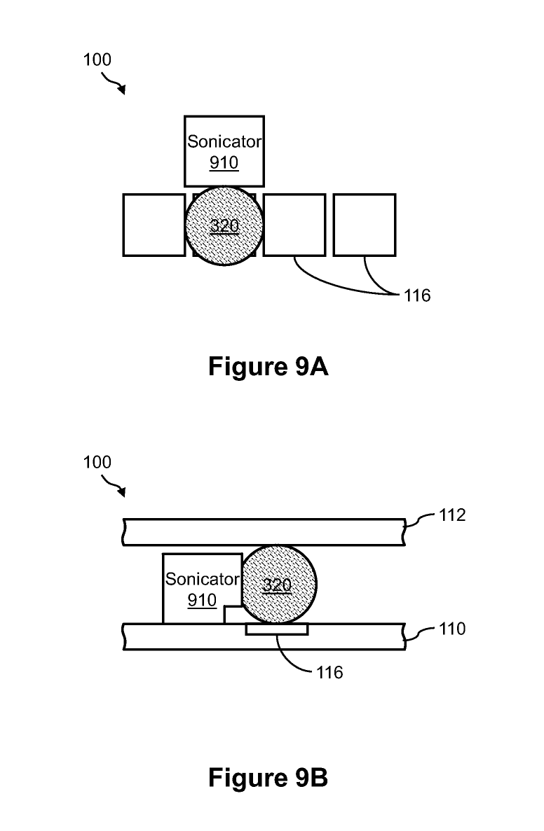

FIGS. 9A and 9B illustrate a top view and another cross-sectional view, respectively, of a portion of droplet actuator 100 of FIG. 1 that includes an on-chip sonication mechanism associated with droplets, thereby promoting cell lysis. In one example, droplet actuator 100 includes an on-chip sonicator 910 that is installed in close proximity to the line or path of droplet operations electrodes 116. In one example, on-chip sonicator 910 may be a surface-mounted sonication device. One or more on-chip sonicators 910 may be implemented on the top and/or bottom substrates. Using droplet operations, a sample droplet 320 may be transported to be in association with on-chip sonicator 910. In one example, sample droplet 320 is directly in contact with on-chip sonicator 910. When on-chip sonicator 910 is activated, ultrasonic energy is transferred from on-chip sonicator 910 to the cell-containing sample droplet 320. By use of on-chip sonicator 910, cell lysis occurs in sample droplet 320.

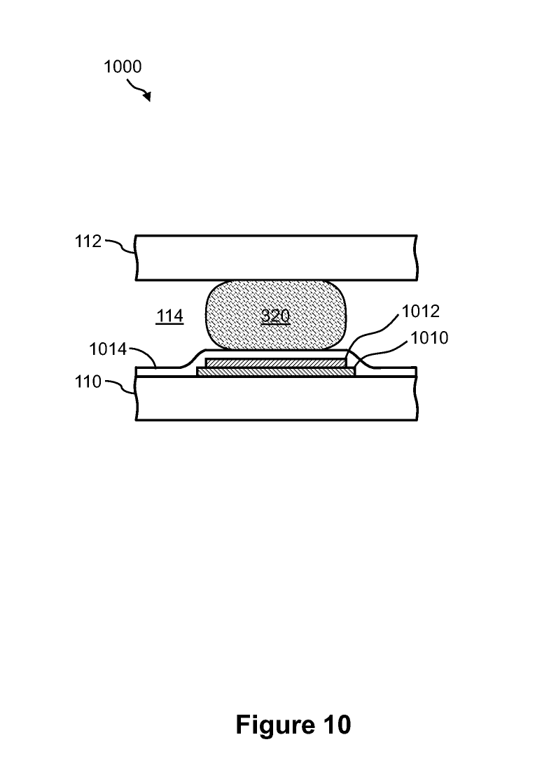

FIG. 10 illustrates a cross-sectional view of an on-chip sonication mechanism 1000, which is yet another example of a sonication mechanism for promoting cell lysis that is integrated into a droplet actuator. In this example, on-chip sonication mechanism 1000 may include piezoelectric film that is patterned directly on the substrate. For example, FIG. 10 shows a piezoelectric film 1010 patterned on bottom substrate 110 of droplet actuator 100 of FIG. 1. Piezoelectric film 1010 may be formed of any piezoelectric material, such as certain crystals and ceramics. One example of piezoelectric material is quartz (Si02). Piezoelectric film 1010 may be used as a source of ultrasonic vibration. Patterned atop piezoelectric film 1010 may be a conductive film 1012, which may serve as a droplet operations electrode for performing droplet operations. Atop conductive film 1012 is a dielectric layer 1014. Dielectric layer 1014 may be, for example, a layer of hydrophobic material. In this configuration, on-chip sonication mechanism 1000 may be driven through the standard droplet operations control lines of droplet actuator 100 to create ultrasonic vibration. Additionally, on-chip sonication mechanism 1000 may be driven through control lines (not shown) that are separate from the standard droplet operations control lines. The structure shown in FIG. 10 is exemplary only. The piezoelectric film 1010, conductive film 1012, and dielectric layer 1014 may be implemented in different orders and configurations, and may be may be incorporated in the bottom substrate, top substrate, and/or both substrates.

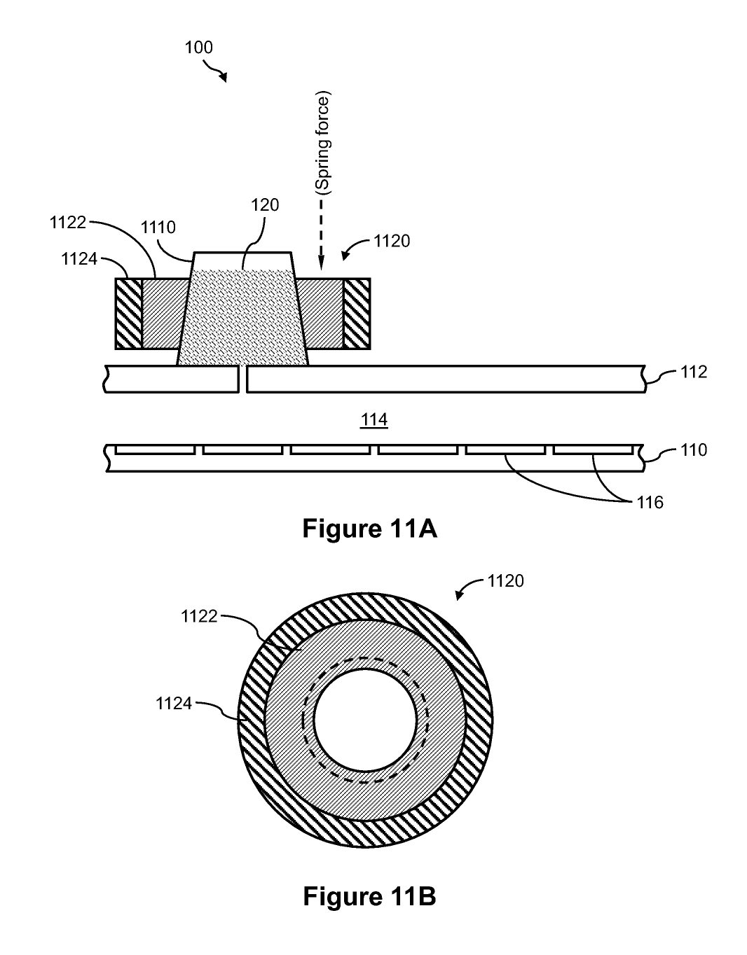

FIG. 11A illustrates another cross-sectional view of a portion of droplet actuator 100 of FIG. 1 in contact with a sonication mechanism for promoting cell lysis. Like FIG. 1, FIG. 11A shows an example of a sonication mechanism in association with a sample reservoir 1110 of a droplet actuator 100. However, in this example, droplet actuator 100 includes a tapered sample reservoir 1110. Tapered sample reservoir 1110, in one example is tapered from narrow to wide from top to bottom, the bottom being the portion of tapered sample reservoir 1110 that interfaces with top substrate 112. A certain quantity of cell-containing sample fluid 120 is held in tapered sample reservoir 1110. A sonication mechanism 1120 is fitted around tapered sample reservoir 1110 for providing ultrasonic energy to the cell-containing sample fluid 120.

FIG. 11B illustrates a top view of an example of sonication mechanism 1120. In this example, sonication mechanism 1120 includes a sonic coupler 1122 for coupling ultrasonic energy from an ultrasonic actuator, such as a lead zirconate titanate (PZT) actuator 1124, to tapered sample reservoir 1110. More specifically, sonic coupler 1122 is fitted inside the tubular or ring PZT actuator 1124. Sonic coupler 1122 may be formed of any material, such as aluminum, that is suitable for conducting ultrasonic energy. The innermost surface of sonic coupler 1122 has substantially the same tapered profile as tapered sample reservoir 1110. The outermost surface of sonic coupler 1122 has substantially the same surface profile as PZT actuator 1124. PZT actuator 1124 may be a tubular or ring ceramic PZT sonic transducer, such as those supplied by Annon Piezo Technology Co., Ltd. (Shenzhen, China). In one example, PZT actuator 1124 has a radial resonance frequency from about 10 kHz to about 50 kHz.

Sonication mechanism 1120 may be closely associated with tapered sample reservoir 1110 to ensure efficient transfer of ultrasonic energy to the cell-containing sample fluid 120 in tapered sample reservoir 1110. In one example, sonication mechanism 1120 may be tightly fitted to tapered sample reservoir 1110 by spring force. Tapered sample reservoir 1110 may be formed, for example, of injection-molded plastic and has thin walls. For example, the walls of tapered sample reservoir 1110 may be about 0.5 mm or less in thickness. When PZT actuator 1124 of sonication mechanism 1120 is activated, ultrasonic energy is transferred to the cell-containing sample fluid 120 and cell lysis occurs.

It shall be appreciated that the sample reservoir may be of various shapes and sizes and the above example of a tampered sample reservoir is, but one non-limiting example of a reservoir configuration suitable for carrying out the invention. For example, the sample reservoir may be substantially cylindrical, square, rectangular, or trapezoidal. Wherein, a sonication mechanism is configured to fit with the sample reservoir to ensure efficient transfer of ultrasonic energy to the contents of the sample reservoir.

### 7.2 Cell Lysis by Heating

Cell lysis methods through cell rupture can be classified into mechanical methods and non-mechanical methods. The use of thermal methods is an example of non-mechanical cell lysis methods. In many cases, heat can promote the cell lysis process and reduce the sample preparation time. Disclosed herein are novel systems, structures, and/or methods of implementing thermally-induced cell lysis and/or for using the combination of heat and sonication in droplet actuators for promoting cell lysis in sample droplets and/or in any volumes of cell-containing sample fluid.

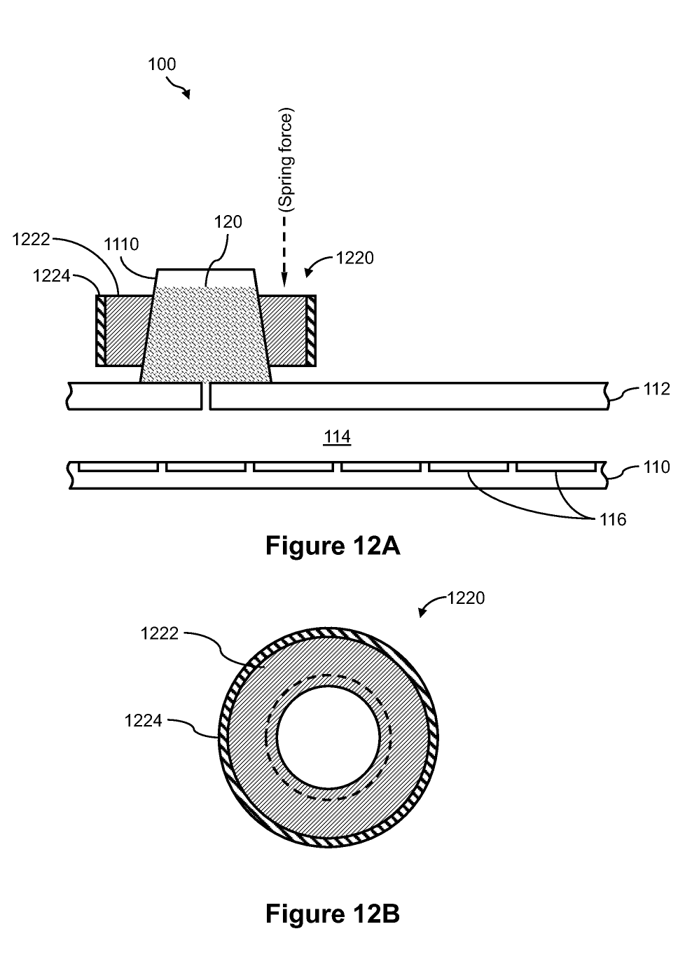

FIG. 12A illustrates yet another cross-sectional view of a portion of droplet actuator 100 of FIG. 1 and shows an example of a heating mechanism or device coupled thereto for promoting cell lysis. In this example, droplet actuator 100 includes tapered sample reservoir 1110 that is described in FIGS. 11A and 11B. A certain quantity of cell-containing sample fluid 120 is held in tapered sample reservoir 1110. A heating mechanism 1220 is fitted around tapered sample reservoir 1110 for providing thermal energy to the cell-containing sample fluid 120.

FIG. 12B illustrates a top view of an example of heating mechanism 1220. In this example, heating mechanism 1220 includes a thermal coupler 1222 for coupling heat energy from a heater 1224 to tapered sample reservoir 1110. More specifically, thermal coupler 1222 is fitted inside the ring-shaped heater 1224. Thermal coupler 1222 may be formed of any thermally conductive material, such as aluminum. The innermost surface of thermal coupler 1222 has substantially the same tapered profile as tapered sample reservoir 1110. Heater 1224 may be a flexible heater ring that is fitted around the outmost surface of thermal coupler 1222. In one example, heater 1224 is a flexible silicone rubber heater, such as those supplied by Minco Products, Inc, (Minneapolis, Minn.).

Heating mechanism 1220 may be closely associated with tapered sample reservoir 1110 to ensure efficient transfer of ultrasonic energy to the cell-containing sample fluid 120 in tapered sample reservoir 1110. In one example, heating mechanism 1220 may be tightly fitted to tapered sample reservoir 1110 by spring force. A thermistor (not shown) may be coupled to thermal coupler 1222 for monitoring the temperature of and controlling heater 1224. When heater 1224 of heating mechanism 1220 is activated, heat energy is transferred to the cell-containing sample fluid 120 and cell lysis occurs.

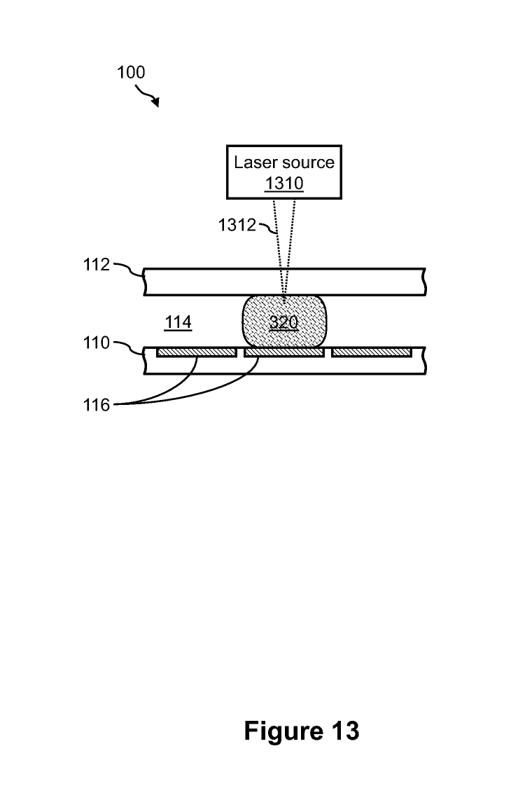

FIG. 13 illustrates yet another cross-sectional view of a portion of droplet actuator 100 of FIG. 1 and shows an example of using a laser as the heat source for promoting cell lysis, FIG. 13 shows a laser source 1310 that is emitting laser energy 1312 through a substrate of droplet actuator 100. In one example, laser energy 1312 is emitted through top substrate 112. In this example, top substrate 112 is substantially transparent to laser energy 1312. Laser source 1310 may be, for example, an infrared (IR) pulsed laser source, or other suitable laser source. In this example, the height of gap 114 of droplet actuator 100 may be about equal to the wavelength (λ) of laser energy 1312 that is emitted by laser source 1310. In another example, the height of gap 114 may be about one half λ). Laser source 1310 for emitting laser energy 1312 is not limited to top substrate 112 only. Laser source 1310 may be incorporated in the bottom substrate, top substrate, and/or both substrates.

When laser source 1310 is activated, laser energy 1312 impinges on the cell-containing sample droplet 320 and causes local heating and pressure pulses to occur therein. The presence of local heating and pressure pulses induces cavitation in sample droplet 320, thereby promoting cell lysis in sample droplet 320.

It shall be appreciated that the sample reservoir may be of various shapes and sizes and the above example of a tampered sample reservoir is, but one non-limiting example of a reservoir configuration suitable for carrying out the invention. For example, the sample reservoir may be substantially cylindrical, square, rectangular, or trapezoidal. Wherein, a heating mechanism is configured to fit with the sample reservoir to ensure efficient transfer of heat energy to the contents of the sample reservoir.

### 7.3 Cell Lysis by the Combination of Sonication and Heat

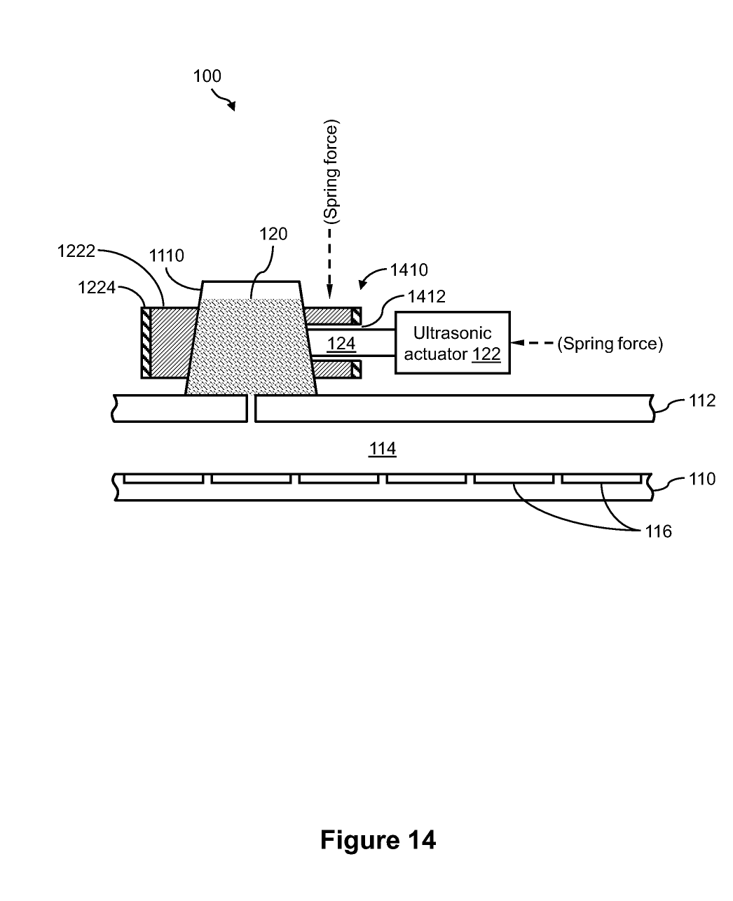

FIG. 14 illustrates yet another cross-sectional view of a portion of droplet actuator 100 of FIG. 1 and shows an example of incorporating the combination of a sonication mechanism and a heating mechanism for promoting cell lysis. In this example, droplet actuator 100 includes tapered sample reservoir 1110 that is described in FIGS. 11A and 11B. A certain quantity of cell-containing sample fluid 120 is held in tapered sample reservoir 1110. A combination mechanism 1410 is fitted in association with tapered sample reservoir 1110 for providing both ultrasonic energy and thermal energy to the cell-containing sample fluid 120. Combination mechanism 1410 may include substantially the same heating mechanism 1220 that is described in FIGS. 12A and 12B, except that a portion of thermal coupler 1222 and heater 1224 has a clearance hole 1412 through which, for example, tip 124 of ultrasonic actuator 122 may be inserted. In this way, tip 124 of ultrasonic actuator 122 may be closely associated with the wall of tapered sample reservoir 1110. In one example, tip 124 of ultrasonic actuator 122 may be pressed by spring force against the wall of tapered sample reservoir 1110. Therefore, when heater 1224 and ultrasonic actuator 122 of combination mechanism 1410 are simultaneously activated, both heat energy and ultrasonic energy are transferred to the cell-containing sample fluid 120 and cell lysis occurs.

It shall be appreciated that the sample reservoir may be of various shapes and sizes and the above example of a tampered sample reservoir is, but one non-limiting example of a reservoir configuration suitable for carrying out the invention. For example, the sample reservoir may be substantially cylindrical, square, rectangular, or trapezoidal. Wherein, a combination mechanism, such as Combination mechanism 1410, is configured to fit with the sample reservoir to ensure efficient transfer of heat and ultrasonic energy to the contents of the sample reservoir.

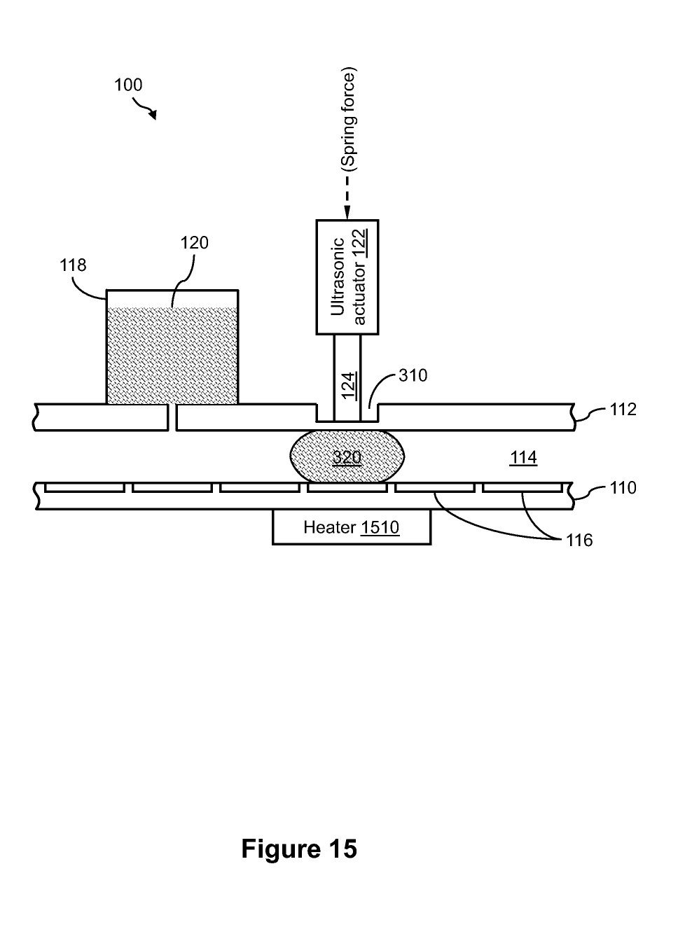

FIG. 15 illustrates yet another cross-sectional view of a portion of droplet actuator 100 of FIG. 1 and shows another example of incorporating the combination of a sonication mechanism and a heating mechanism for promoting cell lysis. FIG. 15 shows substantially the same configuration of droplet actuator 100 that is shown in FIG. 3, which is ultrasonic actuator 122 in association with a substrate of droplet actuator 100, for example top substrate 112. However, FIG. 15 also shows a heater 1510 in thermal contact with the outer surface of an opposing substrate of droplet actuator 100, for example bottom substrate 110. Preferably, heater 1510 is positioned opposite ultrasonic actuator 122. Heater 1510 may be, for example, any type of heater source, such as a heater bar (e.g., a resistance-based heater bar), that is suitable for use with a droplet actuator. When heater 1510 and ultrasonic actuator 122 are simultaneously activated, both heat energy and ultrasonic energy are transferred to the cell-containing sample droplet 320 and cell lysis occurs.

The present invention is not limited to the combinations of sonication and heating that is described with reference to FIGS. 14 and 15. Any combinations of any sonication mechanism and any heating mechanism are possible.

### 7.4 Cell Lysis by Mechanical Shearing

Again, cell lysis methods through cell rupture can be classified into mechanical methods and non-mechanical methods. The use of mechanical shearing methods is another example of mechanical cell lysis methods. Disclosed herein are novel systems, structures, and/or methods of using mechanical shearing in droplet actuators for promoting cell lysis in sample droplets and/or in any volumes of cell-containing sample fluid.

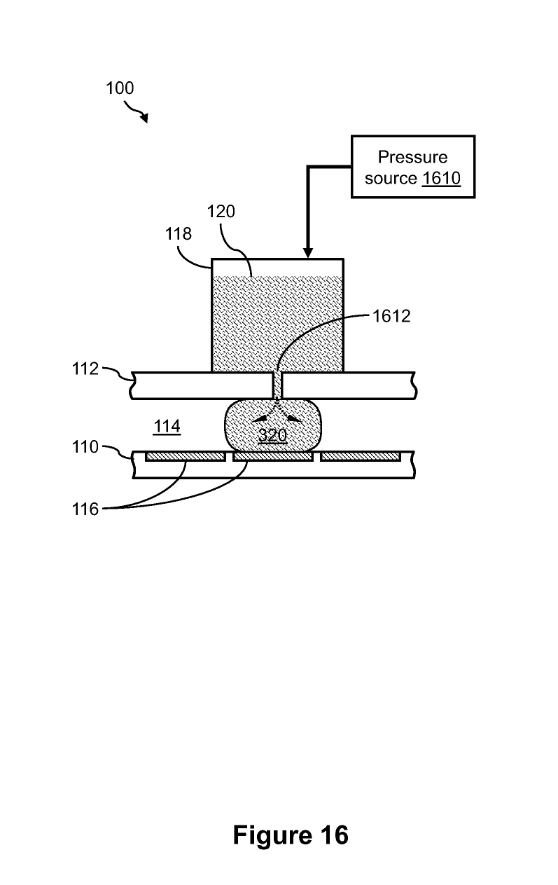

FIG. 16 illustrates yet another cross-sectional view of a portion of droplet actuator 100 of FIG. 1 and shows an example of using mechanical shearing for promoting cell lysis.

In this example, a certain amount of cell-containing sample fluid 120 is provided in sample reservoir 118. Further, a pressure source 1610 connected to sample reservoir 118 is used to force sample fluid 120 into gap 114 of droplet actuator 100 under pressure. In one example, pressure source 1610 is capable of providing pressure at sufficient pounds per square inch (PSI) to cause lysis of cells and/or spores.

At least one opening 1612 in top substrate 112 provides a fluid path from sample reservoir 118 to gap 114 of droplet actuator 100. More specifically, opening 1612 is of suitable size to cause cell disruption due to mechanical shearing when the cell-containing sample fluid 120 is forced under pressure from sample reservoir 118 into gap 114 of droplet actuator 100. Droplet actuator 100 is not limited to one opening 1612 only. Droplet actuator 100 may include any number of small openings 1612 for causing mechanical shearing of the cells in sample fluid 120. Additionally, along with or in place of the one or more openings 1612, cell-containing sample fluid 120 may pass through a filter (not shown) that has a small pore size. Again, when the cell-containing sample fluid 120 is forced under pressure through the filter and into gap 114 of droplet actuator 100, cell disruption and lysing occurs due to mechanical shearing. In any case, due to the mechanical shearing that takes place, one or more lysate droplets 320 may be dispensed from sample reservoir 118.

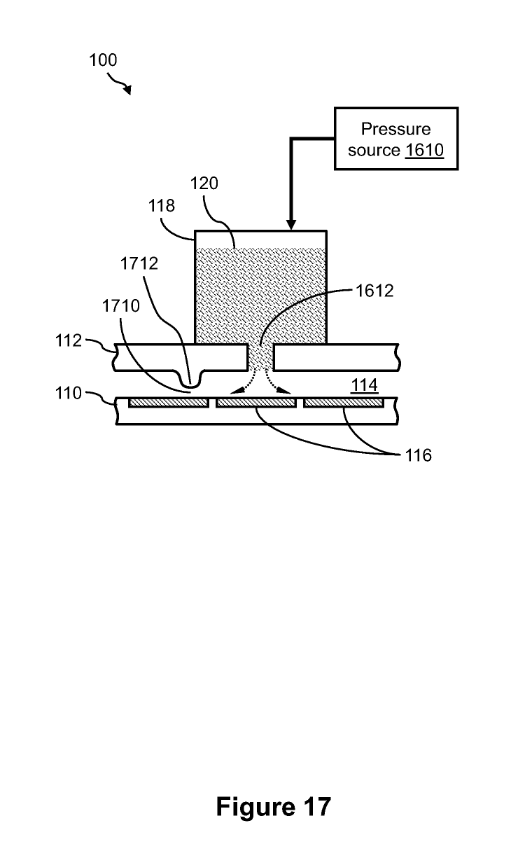

FIG. 17 illustrates yet another cross-sectional view of a portion of droplet actuator 100 of FIG. 1 and shows another example of using mechanical shearing for promoting cell lysis. In this example, the cell-containing sample fluid 120 in sample reservoir 118 is again provided under pressure by use of pressure source 1610. However, in this example, opening 1612 is not necessarily of suitable size to cause cell disruption by mechanical shearing. Instead a narrow opening 1710 is formed in gap 114 of droplet actuator 100. In one example, narrow opening 1710 is formed by a protruded feature 1712 on a surface of one or both of top substrate 112 and/or bottom substrate 110 that is facing gap 114. When formed, narrow opening 1710 is of suitable size to cause cell disruption due to mechanical shearing when the cell-containing sample fluid 120 is forced under pressure therethrough. Droplet actuator 100 is not limited to one narrow opening 1710 only. Any number of protruded features 1712 on the surface of top substrate 112 and/or bottom substrate 110 may be present in droplet actuator 100 to form any number of narrow openings 1710.

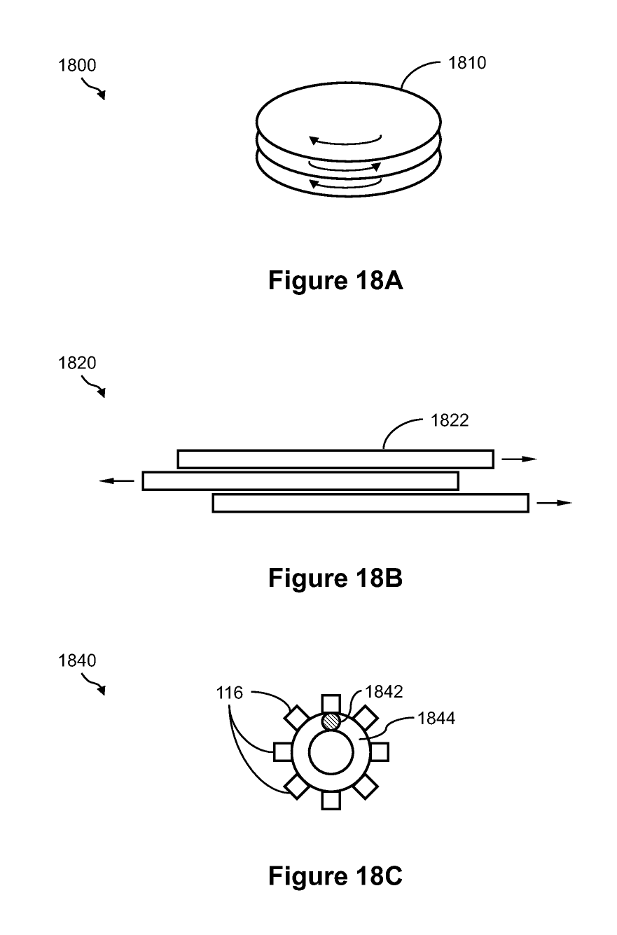

FIGS. 18A, 18B, and 18C illustrate certain views of yet other examples of mechanisms for causing mechanical shearing in a droplet actuator. The mechanisms for causing mechanical shearing in a droplet actuator may be formed by any two or more surfaces that are moving relative to one another. In one example, FIG. 18A shows a disk arrangement 1800 of one or more disks 1810 that may be incorporated in, for example, a sample reservoir and/or in the gap of a droplet actuator. For example, as the cell-containing sample fluid passes around and/or between the one or more disks 1810 that may be spinning, sliding, and/or oscillating, cell disruption may occur by the high shear rates caused by the moving disks 1810. In another example, disk arrangement 1800 may include concentrically-arranged disks.

In another example, FIG. 18B shows a plate arrangement 1820 of one or more plates 1822 that may be incorporated in, for example, a sample reservoir and/or in the gap of a droplet actuator. For example, as the cell-containing sample fluid passes around and/or between the one or more plates 1822 that may be sliding and/or oscillating, cell disruption may occur by the high shear rates caused by the moving plates 1822.

In yet another example, FIG. 18C shows an arrangement 1840 that includes one or more balls 1842, such as metal balls, that are rolling or tumbling in a channel, guide, and/or track 1844. Arrangement 1840 may be incorporated in any environment in which the sample fluid resides, such as in a sample reservoir and/or in the gap of a droplet actuator. For example, FIG. 18C shows a ball 1842 in a channel, guide, and/or track 1844 that is installed in close proximity to an arrangement of droplet operations electrodes 116. As the one or more balls 1842 roll or tumble through the cell-containing sample fluid, cell disruption may occur by the high shear rates caused by the one or more moving balls 1842. The one or more balls 1842 may be moved, for example, magnetically, electrostatically, by pressure differences, by electrowetting, by spinning, and the like.

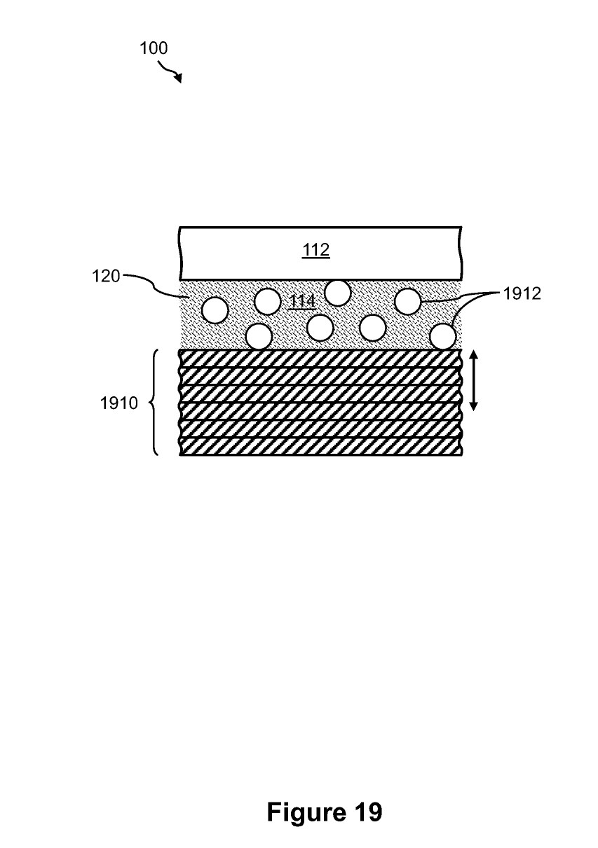

FIG. 19 illustrates another cross-sectional view of a portion of droplet actuator 100 of FIG. 1 and shows another example of using mechanical shearing for promoting cell lysis. In this example, an on-chip piezoelectric stack 1910 is installed in relation to a substrate of droplet actuator 100, for example, top substrate 112. In the gap 114 between piezoelectric stack 1910 and top substrate 112 is a certain quantity of cell-containing sample fluid 120. When piezoelectric stack 1910 is activated, a grinding action occurs in gap 114 between piezoelectric stack 1910 and top substrate 112. The grinding action is due to the ultrasonic vibration of piezoelectric stack 1910, which causes cell lysis to occur in gap 114. Additionally, sample fluid 120 may contain, for example, beads 1912, such as glass or metal beads, to further assist the cell lysis process. For example, when piezoelectric stack 1910 is activated, beads 1912 bounce around in gap 114 due to the ultrasonic vibration and break up cells.

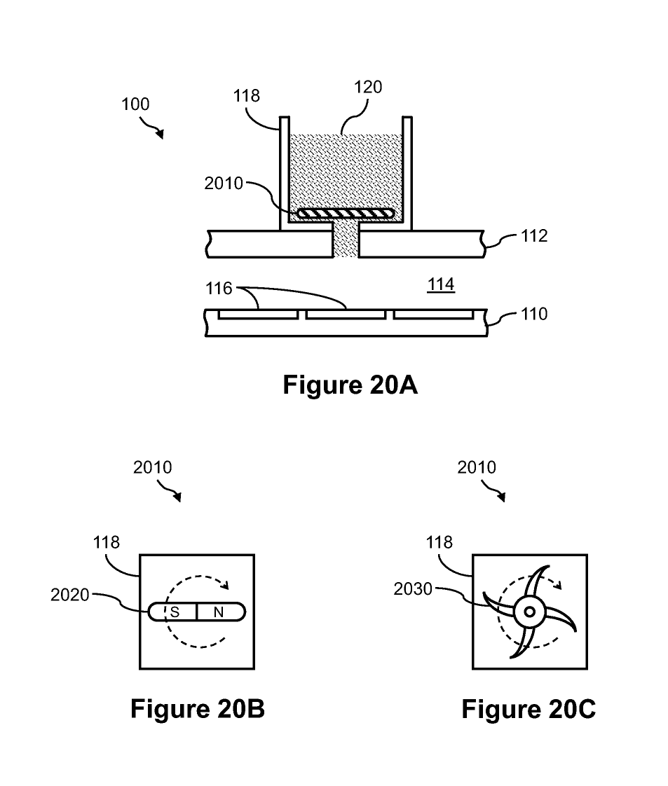

FIG. 20A illustrates yet another cross-sectional view of a portion of droplet actuator 100 of FIG. 1 and shows another example of using mechanical shearing for promoting cell lysis. In this example, a grinding mechanism 2010 is installed in sample reservoir 118 that is holding a quantity of cell-containing sample fluid 120. Grinding mechanism 2010 may be, for example, any grinding mechanism, such as a rotatable grinding mechanism, that is capable of causing cell disruption. By way of example, FIGS. 20B and 20C show two implementations of grinding mechanism 2010.

FIG. 20B illustrates a top view of one example of a grinding mechanism 2010 that is suitable for causing cell disruption. More specifically, FIG. 20B shows grinding mechanism 2010 implemented as a magnetic bar 2020 that is rotatable. The rotating motion of magnetic bar 2020 may be controlled by magnetic forces. The spacing between the magnetic bar 2020 and the floor and/or walls of sample reservoir 118 is suitably small to cause mechanical shearing of the cells when magnetic bar 2020 is in motion.

FIG. 20C illustrates a top view of another example of a grinding mechanism 2010 that is suitable for causing cell disruption. More specifically, FIG. 20C shows grinding mechanism 2010 implemented as a bladed rotor 2030 that is rotatable. Bladed rotor 2030 may be formed of magnetic material. Again, the rotating motion of bladed rotor 2030 may be controlled by magnetic forces, or other suitable mechanism. The spacing between the bladed rotor 2030 and the floor and/or walls of sample reservoir 118 is suitably small to cause mechanical shearing of the cells when bladed rotor 2030 is in motion.

### 7.5 Cell Lysis by Bead Beating

Another mechanical method of cell disruption is referred to as “bead beating.” Current bead beating methods may use glass, ceramic, zirconium, steel, or beads of other suitable material along with a sufficient level of agitation, e.g., by stirring or shaking of the mix. The collisions of beads with cells cause cell disruption. Disclosed herein are novel systems, structures, and/or methods of using bead beating in droplet actuators for promoting cell lysis in sample droplets and/or in any volumes of cell-containing sample fluid.

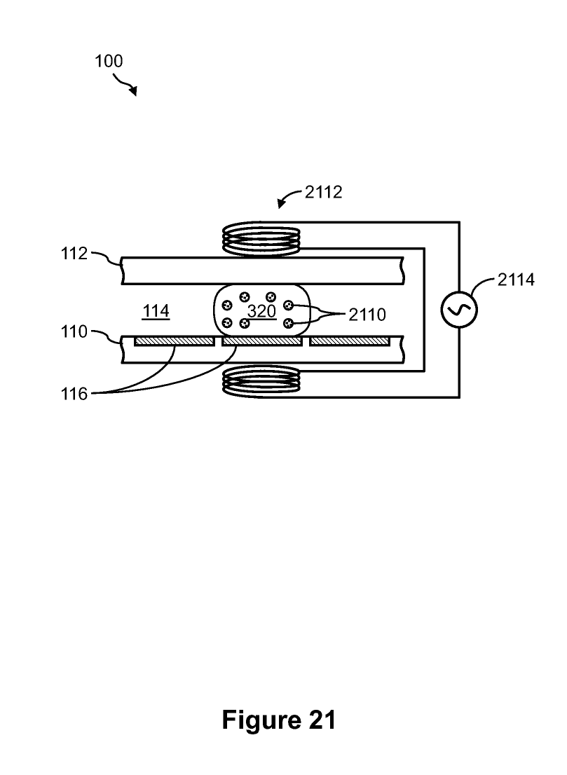

FIG. 21 illustrates yet another cross-sectional view of a portion of droplet actuator 100 of FIG. 1 and shows an example of using electrically-induced bead beating for promoting cell lysis. In this example, magnetically responsive beads 2110 are provided in the cell-containing sample droplet 320. Additionally, a pair of inductors 2112 is installed in close proximity to droplet actuator 100. For example, one inductor 2112 is installed in close proximity to bottom substrate 110 and another inductor 2112 is installed in close proximity to top substrate 112. The pair of inductors 2112 is substantially aligned with a certain droplet operations electrode 116, such that the cell-containing sample droplet 320, which also includes magnetically responsive beads 2110, may be positioned therebetween. A power source 2114 drives the pair of inductors 2112. Power source 2114 is capable of driving inductors 2112 at ultrasonic or near ultrasonic frequency. In one example power source 2114 is an alternating current (AC) power source When inductors 2112 are activated an electrically induced vibration occurs. Consequently, the magnetically responsive beads 2110 are agitated at ultrasonic or near ultrasonic frequency to create a bead beating action and generate ultrasonic cavitation in the cell-containing sample droplet 320. As a result, a cell lysis process occurs in the cell-containing sample droplet 320 due to this bead beating action.

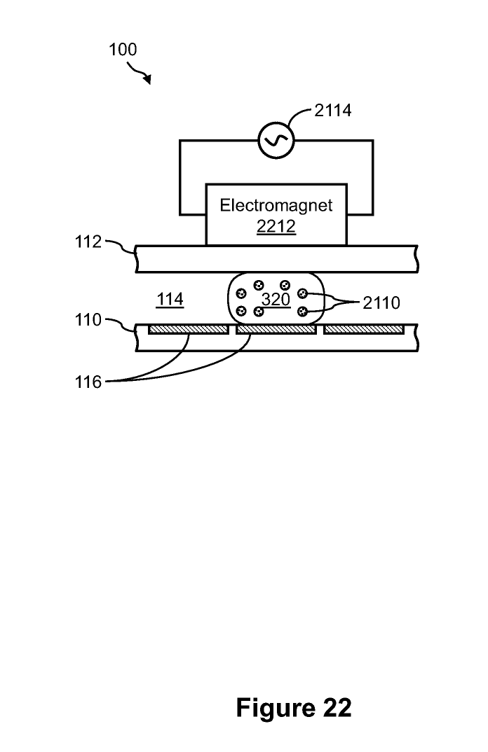

FIG. 22 illustrates yet another cross-sectional view of a portion of droplet actuator 100 of FIG. 1 and shows an example of using magnetically-induced bead beating for promoting cell lysis. Again, magnetically responsive beads 2110 are provided in the cell-containing sample droplet 320. Additionally, an electromagnet 2212 is installed in close proximity to droplet actuator 100. For example, electromagnet 2212 is installed in close proximity to top substrate 112. Alternatively, electromagnet 2212 is installed in close proximity to bottom substrate 110. Electromagnet 2212 is substantially aligned with a certain droplet operations electrode 116. Power source 2114 drives the electromagnet 2212. Power source 2114 is capable of driving electromagnet 2212 at ultrasonic or near ultrasonic frequency. When electromagnet 2212 is activated an electrically-induced vibration occurs. Consequently, the magnetically responsive beads 2110 are agitated at ultrasonic or near ultrasonic frequency to create a bead beating action and generate ultrasonic cavitation in the cell-containing sample droplet 320. As a result, a cell lysis process occurs in the cell-containing sample droplet 320 due to this bead beating action.

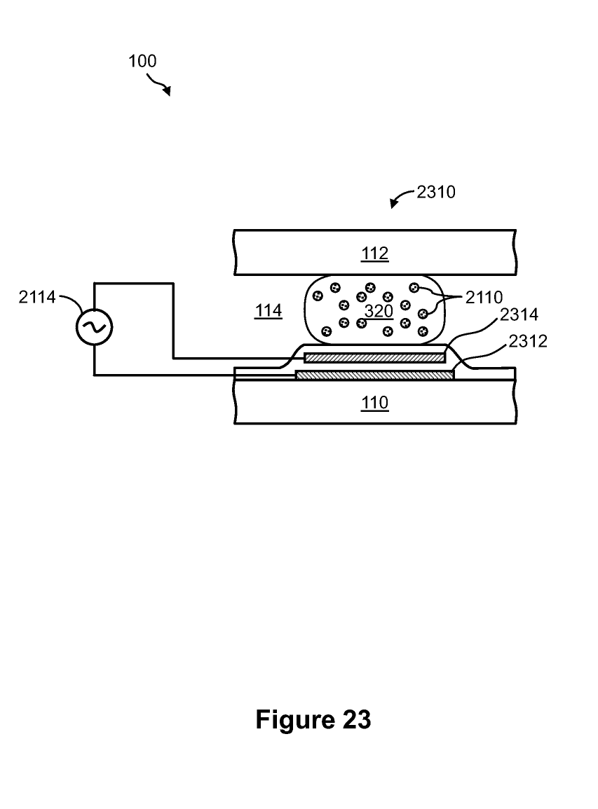

FIG. 23 illustrates yet another cross-sectional view of a portion of droplet actuator 100 of FIG. 1 and shows an example of using electrically-induced bead beating for promoting cell lysis. Again, magnetically responsive beads 2110 are provided in the cell-containing sample droplet 320. Additionally, an electrical structure 2310 for providing an electrically induced vibration is formed on a substrate of droplet actuator 100, for example bottom substrate 110. For example, electrical structure 2310 includes a first conductive plate 2312 and a second conductive plate 2314 that are separated by a dielectric. Power source 2114 is connected between the first conductive plate 2312 and second conductive plate 2314. Power source 2114 is capable of driving electrical structure 2310 at ultrasonic or near ultrasonic frequency. When electrical structure 2310 is activated an electrically-induced vibration occurs. Consequently, the magnetically responsive beads 2110 are agitated at ultrasonic or near ultrasonic frequency to create a bead beating action and generate ultrasonic cavitation in the cell-containing sample droplet 320. As a result, a cell lysis process occurs in the cell-containing sample droplet 320 due to this bead beating action.

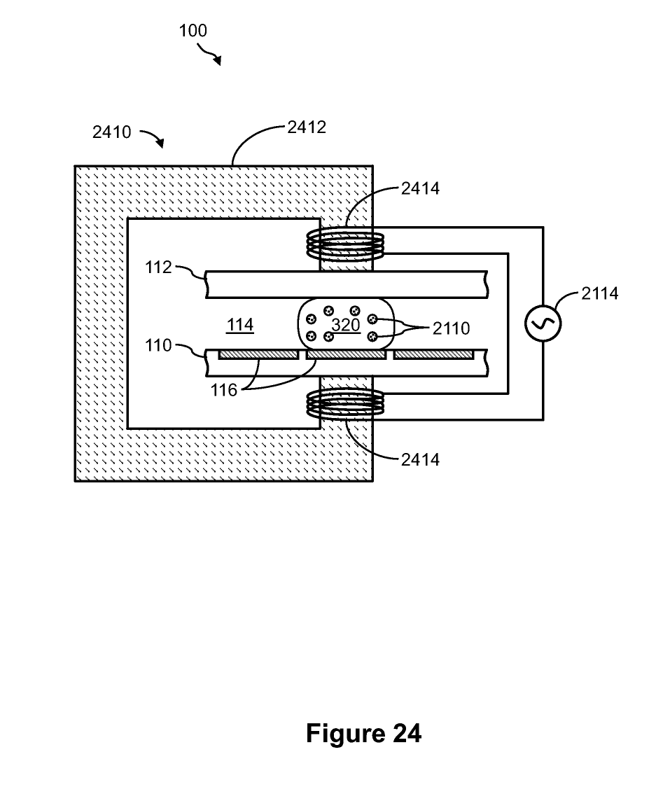

FIG. 24 illustrates yet another cross-sectional view of a portion of droplet actuator 100 of FIG. 1 and shows an example of using magnetically-induced bead beating for promoting cell lysis. Again, magnetically responsive beads 2110 are provided in the cell-containing sample droplet 320 that is positioned at a certain droplet operations electrode 116. Additionally, an electromagnet 2410 is installed in close proximity to droplet actuator 100. For example, electromagnet 2410 includes a shaped magnetic core 2412 (e.g., horseshoe-shaped). Droplet actuator 100 is positioned within the shaped magnetic core 2412, as shown in FIG. 24.

Additionally, electromagnet 2410 includes a pair of inductors 2414 installed in close proximity to droplet actuator 100. For example, one inductor 2414 is installed in close proximity to bottom substrate 110 and another inductor 2414 is installed in close proximity to top substrate 112. The pair of inductors 2414 is substantially aligned with a certain droplet operations electrode 116, such that the cell-containing sample droplet 320, which also includes magnetically responsive beads 2110, may be positioned there between. Power source 2114 drives the pair of inductors 2414. AC power source 2114 is capable of driving inductors 2414 at ultrasonic or near ultrasonic frequency. When inductors 2414 are activated, a magnetically-induced vibration occurs. Consequently, the magnetically responsive beads 2110 are agitated at ultrasonic or near ultrasonic frequency to create a bead beating action and generate ultrasonic cavitation in the cell-containing sample droplet 320. As a result, a cell lysis process occurs in the cell-containing sample droplet 320 due to this bead beating action.

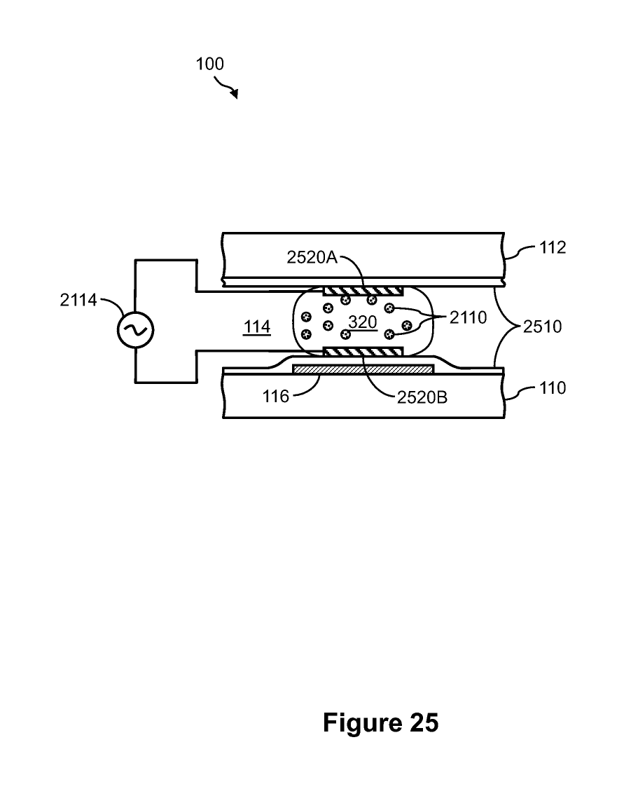

FIG. 25 illustrates yet another cross-sectional view of a portion of droplet actuator 100 of FIG. 1 and shows another example of using electrically-induced bead beating for promoting cell lysis. Again, magnetically responsive beads 2110 are provided in the cell-containing sample droplet 320 that is at a certain droplet operations electrode 116. FIG. 25 shows a dielectric layer 2510 (e.g., a hydrophobic coating) atop the surface of bottom substrate 110 that is facing gap 114. Another dielectric layer 2510 is atop the surface of top substrate 112 that is facing gap 114. Additionally, a pair of electrodes 2520 is arranged in gap 114 of droplet actuator 100, near a certain droplet operations electrode 116. For example, an electrode 2520A is arranged atop dielectric layer 2510 at top substrate 112, and an electrode 2520B is arranged atop dielectric layer 2510 at bottom substrate 110. Power source 2114 is connected between electrode 2520A and electrode 2520B. Power source 2114 is capable of driving electrodes 2520 at ultrasonic or near ultrasonic frequency. When cell-containing sample droplet 320 is at droplet operations electrode 116 and power source 2114 is activated an electrically-induced vibration occurs between electrode 2520A and electrode 2520B. Consequently, the magnetically responsive beads 2110 are agitated at ultrasonic or near ultrasonic frequency to create a bead beating action and generate ultrasonic cavitation in the cell-containing sample droplet 320. As a result, a cell lysis process occurs in the cell-containing sample droplet 320 due to this bead beating action.

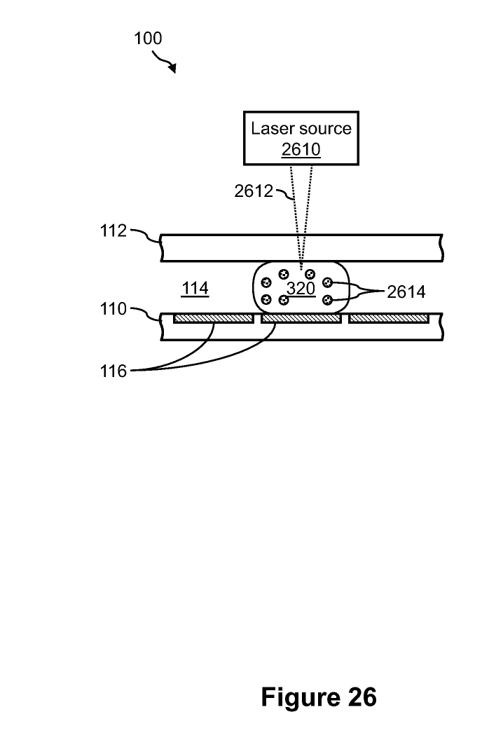

FIG. 26 illustrates yet another cross-sectional view of a portion of droplet actuator 100 of FIG. 1 and shows an example of using laser-assisted bead beating for promoting cell lysis. FIG. 26 shows a laser source 2610 that is emitting laser energy 2612 through a substrate of droplet actuator 100, for example top substrate 112. In this example, top substrate 112 is substantially transparent to laser energy 2612. Laser source 2610 may be, for example, a high power visible laser source. Laser energy 2612 may be pulsed or continuous. A cell-containing sample droplet 320 is at a certain droplet operations electrode 116 and contains certain particles and/or beads 2614.

When laser source 2610 is activated, laser energy 2612 impinges on and heats the particles and/or beads 2614 in the cell-containing sample droplet 320. The particles and/or beads 2614 are heated without necessarily heating the sample liquid. The heated particles and/or beads 2614 agitate the sample liquid to induce collisions between the particles and/or beads 2614 and the cells, thereby causing cell lysis to occur in sample droplet 320.

### 7.6 Ultrasonic Cavitation

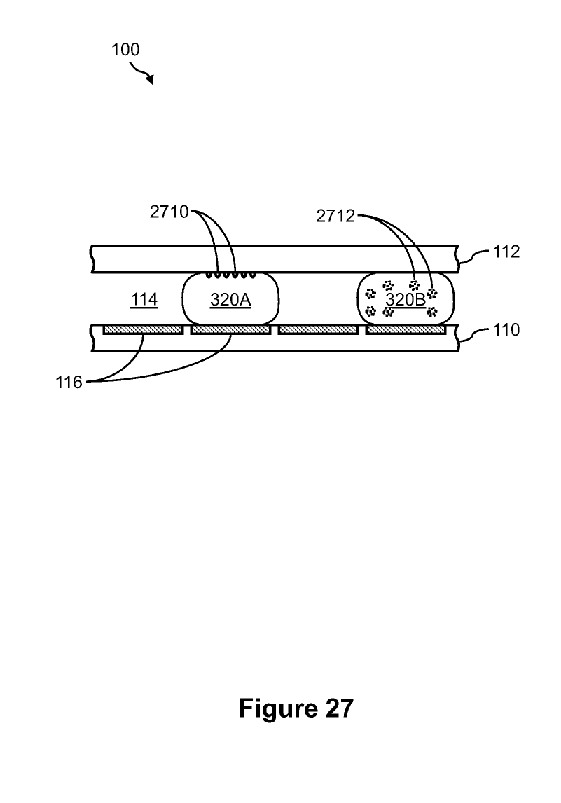

FIG. 27 illustrates yet another cross-sectional view of a portion of droplet actuator 100 of FIG. 1 and shows an example of features incorporated therein that promote ultrasonic cavitation and, thereby, promote cell lysis. Ultrasonic cavitation can occur by incorporating features into droplet actuator 100 that have different acoustic impedances. In one example, certain rough features 2710 may be incorporated on the surface of the top substrate 112 and/or bottom substrate 110 that is facing gap 114. In one example, rough features 2710 may be created by changing the properties of the hydrophobic coating on the substrates. Otherwise, rough features 2710 may be patterned on the surface of the substrates by any suitable means. Rough features 2710 may be used in combination with any of the aforementioned sonication mechanisms disclosed herein. When sonication occurs, bubbles form in, for example, a cell-containing sample droplet 320A due to the presence of these rough features 2710.

Instead of being present on the surfaces of the substrates, certain features to promote ultrasonic cavitation may be present on beads and/or other particulate in the cell-containing sample solution. For example, FIG. 27 shows a cell-containing sample droplet 320B that includes one or more beads 2712. The surface of beads 2712 is rough in nature. Again, when sonication occurs, bubbles form in, for example, a cell-containing sample droplet 320B due to the presence of these rough beads 2712.

Other ways of promoting ultrasonic cavitation in droplet actuators include the use of contrast agents (not shown) in the cell-containing sample fluid. The presence of contrast agents in the sample fluid increases the amount of gas in the solvent, which promotes ultrasonic cavitation. Examples of contrast agents include, but are not limited to, ALBUNEX® and OPTISOWM, both supplied by Mallinckrodt Inc. (St. Louis, Mo.).

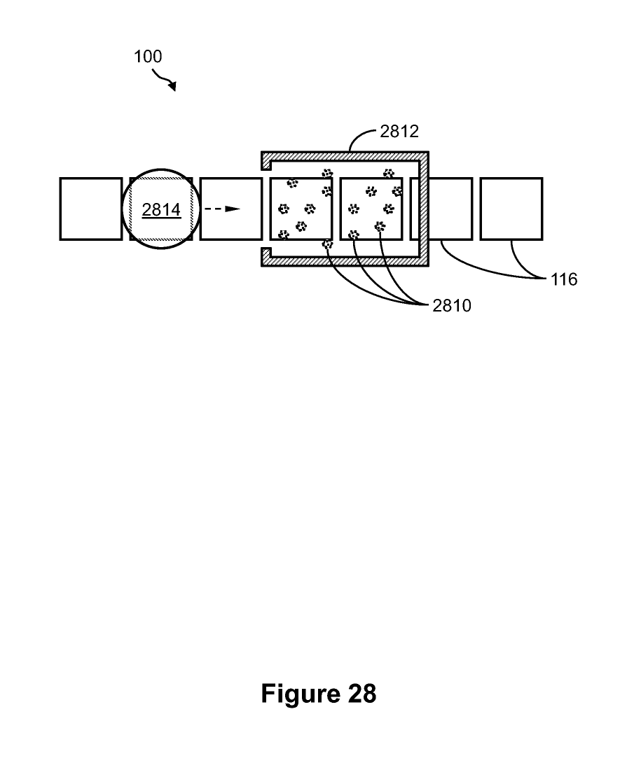

FIG. 28 illustrates another top view of an example of a portion of droplet actuator 100 of FIG. 1 that includes a barrier for retaining microemulsion droplets that may result from sonication and a process of collecting the microemulsion droplets. FIG. 28 shows an arrangement of droplet operations electrodes 116. A microemulsion, such as microemulsion droplets 2810, may be created as a result of, for example, sonication. In this case, sonication can take place in an enclosed area on the droplet actuator in order to keep the microemulsion confined to one area. For example, arranged at certain droplet operations electrodes 116 of droplet actuator 100 is a barrier 2812 that may be used to confine microemulsion droplets 2810. Using droplet operations, a larger droplet, such as a droplet 2814, may be transported into the confines of barrier 2812 to collect the smaller microemulsion droplets 2810 for further processing. Because foaming can occur during sonication, droplet 2814 may include certain anti-foaming agents to reduce the foaming. An example of an anti-foaming agent is silicon oil.

### 7.7 Electrically-Induced Cell Lysis

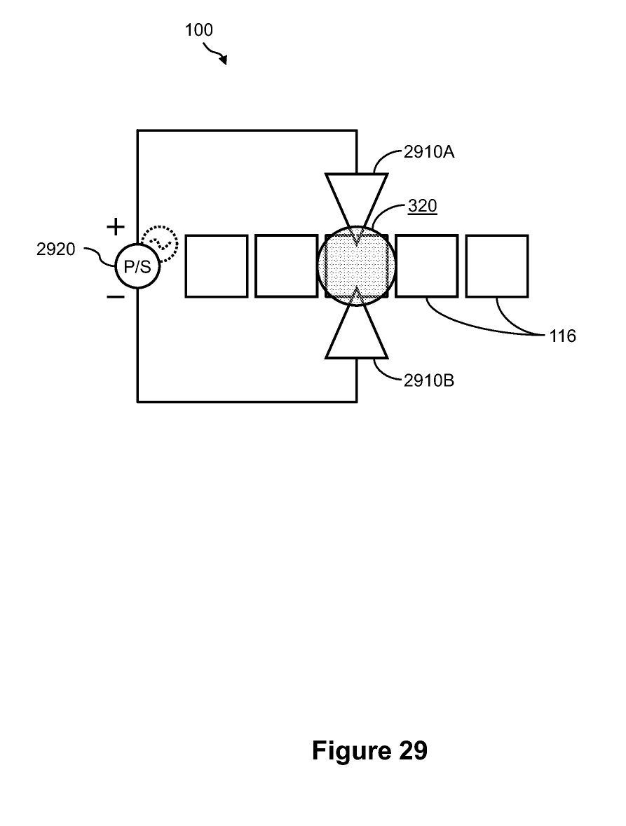

FIG. 29 illustrates yet another top view of a portion of droplet actuator 100 of FIG. 1 and shows an example of using electric fields for promoting cell lysis. In this example, a pair of electrodes 2910 (e.g., electroporation electrodes) is arranged in relation to one or more droplet operations electrodes 116. For example, an electrode 2910A is arranged near one side of a certain droplet operations electrode 116, and an electrode 2910B is arranged near the opposite side of the same droplet operations electrode 116. Electrodes 2910 may be, for example, patterned on the dielectric layer (not shown) of bottom substrate 110, which may be a PCB. Electrodes 2910 may be, for example, positioned on top substrate 112. The present invention is not limited to one pair of electrodes 2910. Any number of pairs of electrodes 2910 may be present along the line of droplet operations electrodes 116. Further, the shape of electrodes 2910 is not limited to that shown in FIG. 29, any shape that is suitable for contacting sample droplet 320 is possible.

A power source 2920 drives the pair of electrodes 2910. Power source 2920 may be an AC and/or direct current (DC) power source. In one example, power source 2920 may be capable of providing a field strength of about 1000 volts per centimeter. Scaled to meet the requirements of droplet actuator 100, power source 2920 may be capable of providing a field strength of about 100 volts per millimeter.

A cell-containing sample droplet 320 is transported via droplet operations between electrodes 2910A and 2910B and, thus, sample droplet 320 is coupled to electrodes 2910A and 2910B. When power source 2920 is activated a high-voltage electric field is created between electrodes 2910A and 2910B. Consequently, current flows through sample droplet 320, which may cause the walls of the cells therein to rupture, thereby causing cell lysis to occur in sample droplet 320.

A side effect of using electric fields for promoting cell lysis in a droplet actuator is that if the metal surfaces (e.g., of electrodes 2910 and/or droplet operations electrodes 116) are not protected they may become fouled by biological material. One could use dielectric material to prevent the metal surfaces from fouling, but this may reduce the charge considerably to the point where it may not be effective for cell lysis. A surface coating of self-assembled monolayers (SAM) provides a suitable protection mechanism to prevent the metal surfaces from fouling while still allowing the full electronic current/potential to be achieved during electrical sample lysis. Examples of SAMs include, but are not limited to, alkane thiols or modified alkane thiols on gold, and alkyl phosphinates or modified alkyl phosphinates on ITO. Both of these metal/alloys (i.e., gold and ITO metal/alloys) can be used to coat any surface into which biological material can be placed. Additionally, both of these metal/alloys allow the full electronic current/potential to be achieved, which will lyse the material and not foul the metal surfaces.

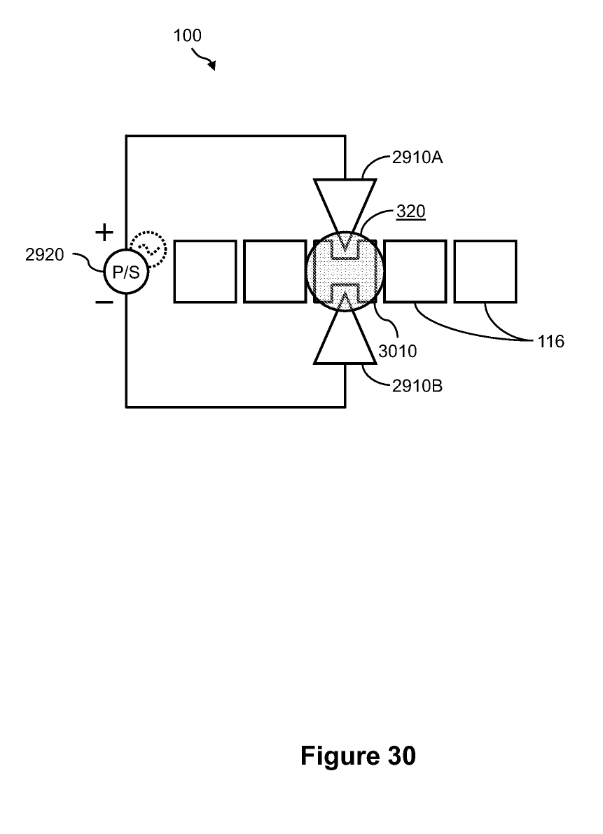

FIG. 30 illustrates yet another top view of a portion of droplet actuator 100 of FIG. 1 and shows another example of using electric fields for promoting cell lysis. The electrode arrangement shown in FIG. 30 is substantially the same as the electrode arrangement shown in FIG. 28, except that the droplet operations electrode 116 between electrodes 2910A and 2910B is replaced with a droplet operations electrode 3010. Droplet operations electrode 3010 includes clearance regions that allow the tips of electrodes 2910A and 2910B to be patterned in the same plane as droplet operations electrode 3010, with no overlap therebetween. The shapes of electrodes 2910 and droplet operations electrode 3010 are not limited to those shown in FIG. 30. Any shapes that allow electrodes 2910 to be patterned in the same plane as droplet operations electrode 3010 are possible.

As described with respect to FIG. 29, the metal surfaces of droplet actuator 100 may have a SAM surface coating to prevent metal fouling while still allowing the full electronic current/potential to be achieved during electrical sample lysis.

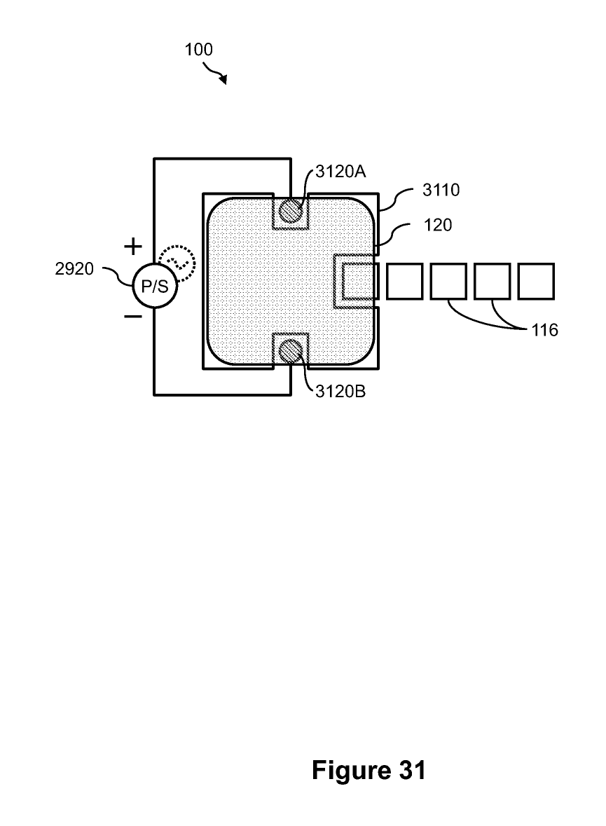

FIG. 31 illustrates yet another top view of a portion of droplet actuator 100 of FIG. 1 and shows another example of using electric fields for promoting cell lysis. In this example, a reservoir electrode 3110 is arranged in relation to a line or path of droplet operations electrodes 116 of droplet actuator 100. A quantity of sample fluid 120 may be present at reservoir electrode 3110. Reservoir electrode 3110 may include a pair of clearance regions on each side thereof in which a corresponding pair of electrodes 3112 (e.g., electroporation electrodes). For example, an electrode 3112A is arranged at one side of reservoir electrode 3110, and an electrode 3112B is arranged at the opposite side of reservoir electrode 3110. In one example, electrodes 3112 may be vertical solder posts that are installed, for example, in the PCB. In another example, electrodes 3112 may be vias in the PCB. Again, power source 2920, which is described with reference to FIG. 29, may be driving electrodes 3112. When power source 2920 is activated a high-voltage electric field is created between electrodes 3112A and 3112B. Consequently, current flows through sample fluid 120, which may cause the walls of the cells therein to rupture, thereby causing cell lysis to occur in sample fluid 120.

As described with respect to FIG. 29, the metal surfaces of droplet actuator 100 may have a SAM surface coating to prevent metal fouling while still allowing the full electronic current/potential to be achieved during electrical sample lysis.

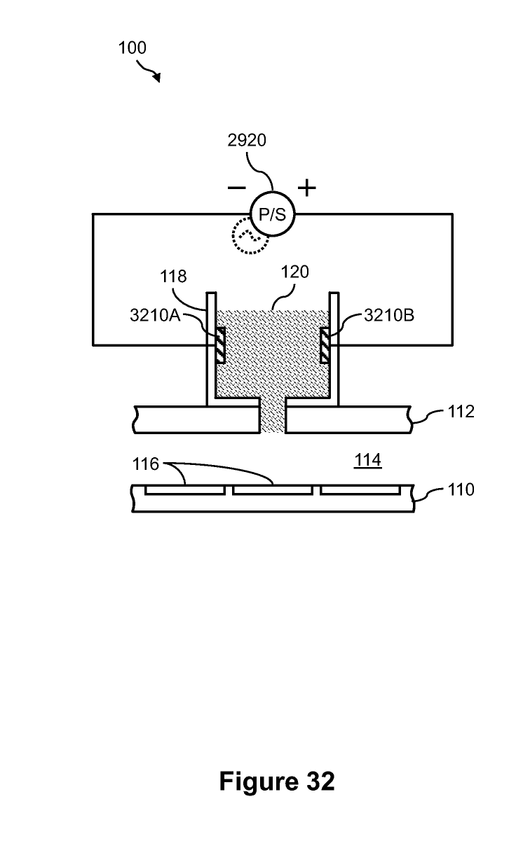

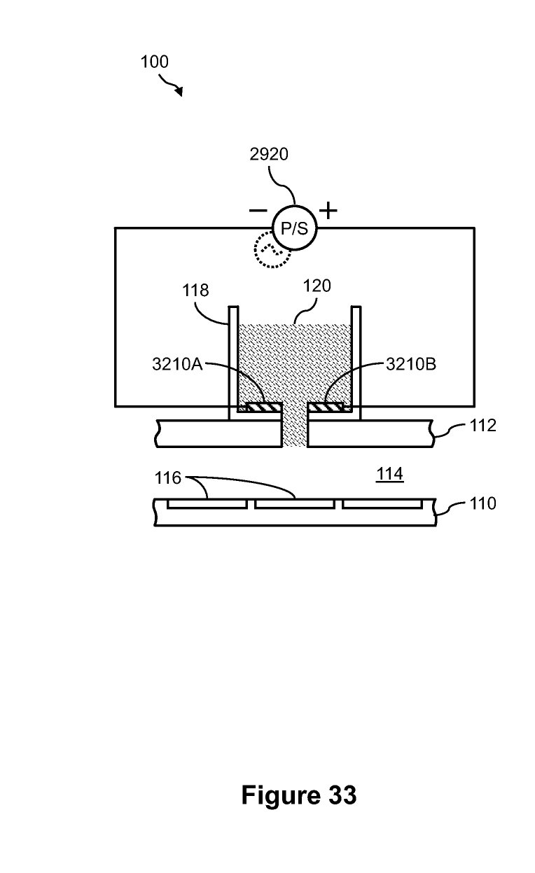

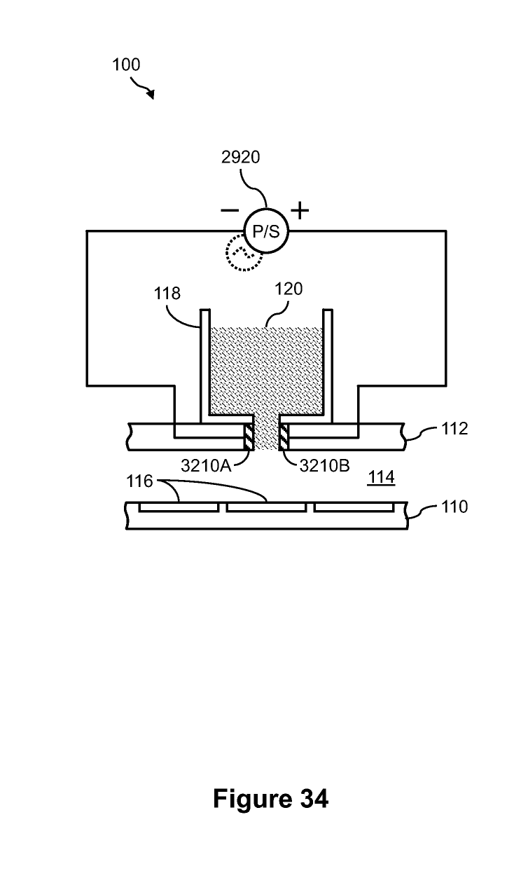

FIG. 32 illustrates yet another cross-sectional view of a portion of droplet actuator 100 of FIG. 1 and shows another example of using electric fields for promoting cell lysis. In this example, a pair of electrodes 3210 (e.g., electroporation electrodes) is positioned in sample reservoir 118 that is holding a quantity of cell-containing sample fluid 120. For example, an electrode 3210A is arranged on one sidewall of sample reservoir 118, and an electrode 3210B is arranged on an opposing sidewall of sample reservoir 118. The present invention is not limited to one pair of electrodes 3210. Any number of pairs of electrodes 3210 may be present along the sidewalls of sample reservoir 118. Again, power source 2920, which is described with reference to FIG. 29, may be driving electrodes 3210. When power source 2920 is activated a high-voltage electric field is created between electrodes 3210A and 3210B. Consequently, current flows through sample fluid 120, which may cause the walls of the cells therein to rupture, thereby causing cell lysis to occur in the bulk sample fluid 120. Other positions of electrodes 3210 in and/or near sample reservoir 118 are possible; examples of which are shown in FIGS. 33 and 34.

As described with respect to FIG. 29, the metal surfaces of droplet actuator 100 may have a SAM surface coating to prevent metal fouling while still allowing the full electronic current/potential to be achieved during electrical sample lysis.

FIG. 33 illustrates yet another cross-sectional view of a portion of droplet actuator 100 of FIG. 1 and shows another example of using electric fields for promoting cell lysis. In this example, the pair of electrodes 3210 is positioned at the floor of sample reservoir 118 and in proximity an opening that leads to gap 114 of droplet actuator 100. Again, power source 2920 may be driving electrodes 3210. When power source 2920 is activated a high-voltage electric field is created between electrodes 3210A and 3210B. Consequently, current flows through sample fluid 120, which may cause the walls of the cells therein to rupture. In this embodiment, cell lysis occurs in a localized portion of sample fluid 120. More specifically, cell lysis occurs in sample fluid 120 as the flow approaches the opening that leads from sample reservoir 118 to gap 114 of droplet actuator 100, rather than in the bulk sample fluid 120.

As described with respect to FIG. 29, the metal surfaces of droplet actuator 100 may have a SAM surface coating to prevent metal fouling while still allowing the full electronic current/potential to be achieved during electrical sample lysis.

FIG. 34 illustrates yet another cross-sectional view of a portion of droplet actuator 100 of FIG. 1 and shows another example of using electric fields for promoting cell lysis. In this example, the pair of electrodes 3210 is positioned along the walls of the opening that leads from sample reservoir 118 to gap 114 of droplet actuator 100. Again, power source 2920 may be driving electrodes 3210. When power source 2920 is activated a high-voltage electric field is created between electrodes 3210A and 3210B. Consequently, current flows through sample fluid 120, which may cause the walls of the cells therein to rupture. In this embodiment, cell lysis occurs in a localized portion of sample fluid 120. More specifically, cell lysis occurs in sample fluid 120 at it flows through the opening that leads from sample reservoir 118 to gap 114 of droplet actuator 100, rather than in the bulk sample fluid 120.

As described with respect to FIG. 29, the metal surfaces of droplet actuator 100 may have a SAM surface coating to prevent metal fouling while still allowing the full electronic current/potential to be achieved during electrical sample lysis.

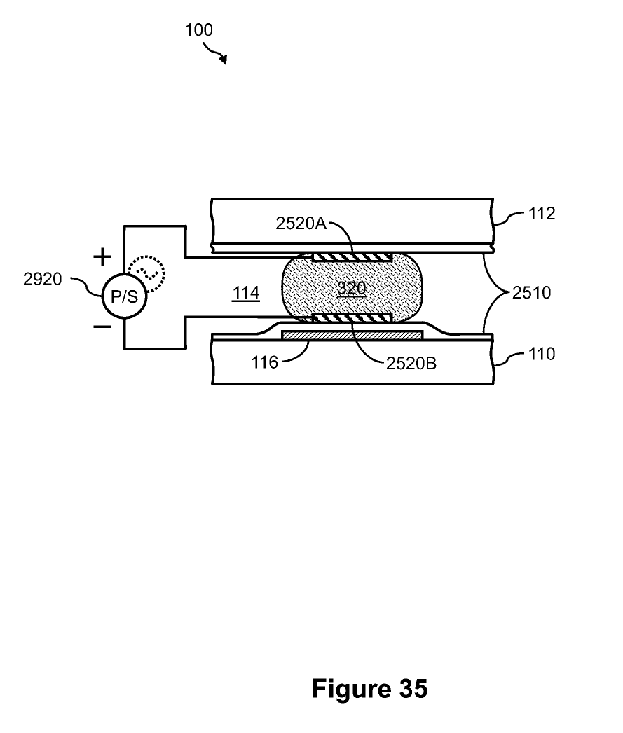

FIG. 35 illustrates yet another cross-sectional view of a portion of droplet actuator 100 of FIG. 1 and shows another example of using electric fields for promoting cell lysis. In this example, FIG. 35 shows substantially the same arrangement of electrodes 2520A and 2520B at the top substrate 112 and bottom substrate 110, respectively, of droplet actuator 100 as described with reference to FIG. 25. However, in this example, electrodes 2520A and 2520B are driven by power source 2920 instead of power source 2114. Additionally, the cell-containing sample droplet 320 does not necessarily include magnetically responsive beads 2110. When power source 2920 is activated a high-voltage electric field is created between electrodes 2520A and 2520B. Consequently, current flows through sample droplet 320, which may cause the walls of the cells therein to rupture, thereby causing cell lysis to occur in sample droplet 320.

As described with respect to FIG. 29, the metal surfaces of droplet actuator 100 may have a SAM surface coating to prevent metal fouling while still allowing the full electronic current/potential to be achieved during electrical sample lysis.

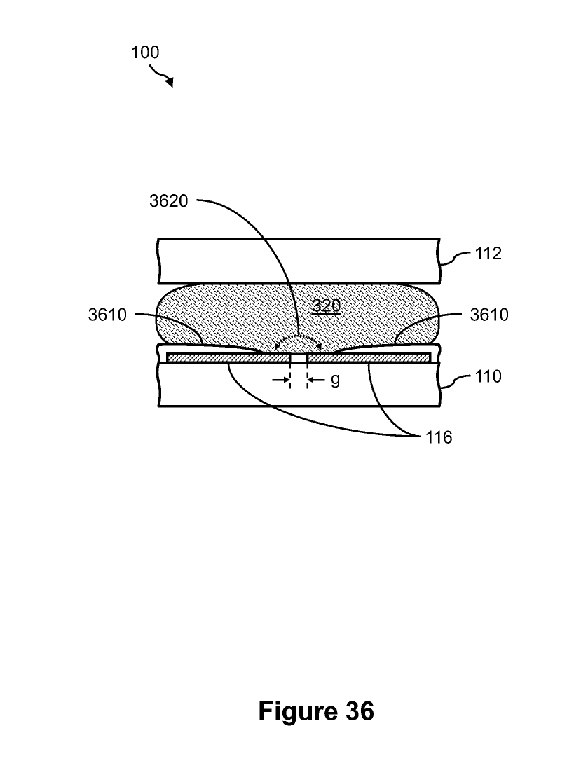

FIG. 36 illustrates yet another cross-sectional view of a portion of droplet actuator 100 of FIG. 1 and shows another example of using electric fields for promoting cell lysis.

FIG. 36 shows a dielectric layer 3610 (e.g., a hydrophobic coating) atop droplet operations electrodes 116 of bottom substrate 110. A selective portion of dielectric layer 3610 is absent along bottom substrate 110, thereby exposing selective portions of adjacent droplet operations electrodes 116, as shown in FIG. 36. These exposed portions of adjacent droplet operations electrodes 116 may be used as electrodes for performing electrically-induced cell lysis in gap 114 of droplet actuator 100.

In this example, droplet operations electrodes 116 may be used for the dual purpose of performing droplet operations and performing electrically-induced cell lysis. For example, the control lines that are used for controlling droplet operations are also used to apply voltage at the exposed portions of adjacent droplet operations electrodes 116 for promoting cell lysis. The gap between the adjacent droplet operations electrodes 116 is suitably small that an electric field 3620 is created between the exposed portions of the adjacent droplet operations electrodes 116 when a voltage is applied. Consequently, current flows through sample droplet 320, which may cause the walls of the cells therein to rupture, thereby causing cell lysis to occur in sample droplet 320. Droplet operations electrodes 116 are not limited to bottom substrate 110, and may be present on either, or both, of top substrate 112 and/or bottom substrate 110.

As described with respect to FIG. 29, the metal surfaces of droplet actuator 100 may have a SAM surface coating to prevent metal fouling while still allowing the full electronic current/potential to be achieved during electrical sample lysis.

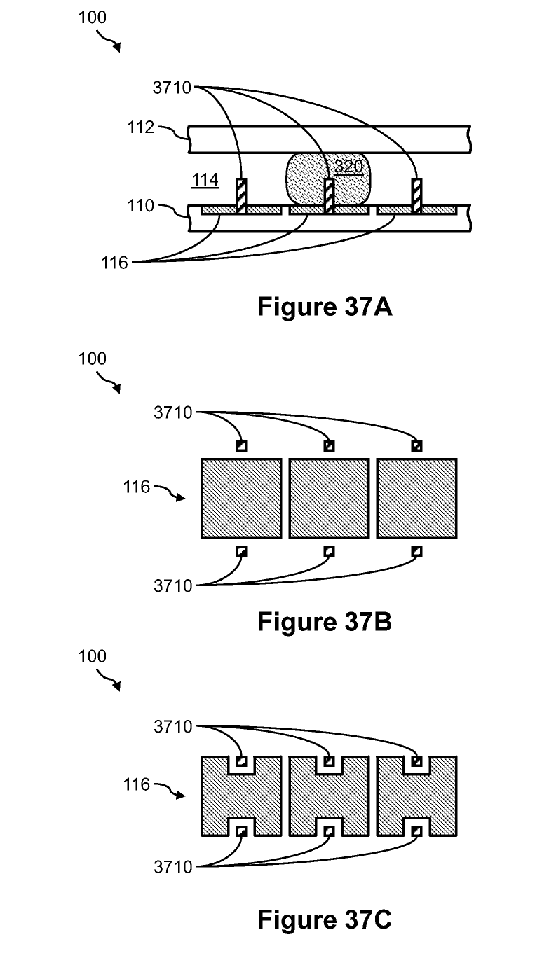

FIGS. 37A, 37B, and 37C illustrate yet other cross-sectional and top views of a portion of droplet actuator 100 of FIG. 1 and show another example of using electric fields for promoting cell lysis. For example, FIG. 37A (cross-sectional view) and FIG. 37B (top view) show an arrangement of electrodes 3710 (e.g., electroporation electrodes) alongside the line and/or path of droplet operations electrodes 116. In an alternative arrangement, FIG. 37C shows clearance regions in droplet operations electrodes 116 in which electrodes 3710 may be inset.

Electrodes 3710 may have a certain height that extends into gap 114 of droplet actuator 100. In one example, electrodes 3710 are implemented by solder posts alongside of droplet operations electrodes 116. Using droplet operations, a cell-containing sample droplet 320 may be transported along droplet operations electrodes 116. At each droplet operations electrode 116 the cell-containing sample droplet 320 comes into contact with a pair of opposing electrodes 3710. Again, power source 2920 (not shown) may be driving electrodes 3710. When power source 2920 is activated a high-voltage electric field is created between opposing electrodes 3710. Consequently, current flows through sample droplet 320, which may cause the walls of the cells therein to rupture, thereby causing cell lysis to occur in the bulk sample droplet 320.

As described with respect to FIG. 29, the metal surfaces of droplet actuator 100 may have a SAM surface coating to prevent metal fouling while still allowing the full electronic current/potential to be achieved during electrical sample lysis.

### 7.8 Cell Lysis by Thermal Cycling

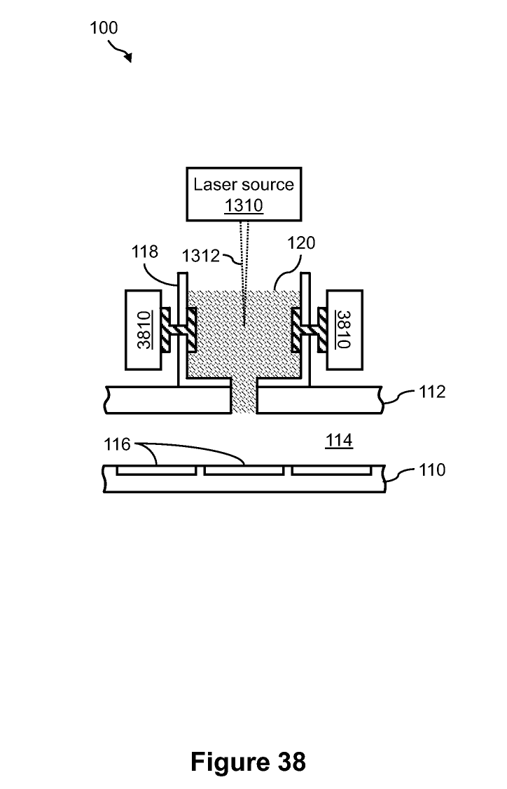

FIG. 38 illustrates yet another cross-sectional view of a portion of droplet actuator 100 of FIG. 1 and shows an example of using thermal cycling for promoting cell lysis. In this example, a thermoelectric module 3810 is in thermal contact with the walls of sample reservoir 118. In one example, thermoelectric module 3810 may include a Peltier cooler. Sample reservoir 118 contains a quantity of cell-containing sample fluid 120. Additionally, a heat source, such as, but not limited to, laser source 1310 of FIG. 13 and/or laser source 2610 of FIG. 26, may be positioned at sample reservoir 118 for emitting laser energy 1312 into the cell-containing sample fluid 120. Thermoelectric module 3810 is the cooling source, while laser source 1310 is the heat source. By coordinating the operations of thermoelectric module 3810 with the operations of laser source 1310, a freeze-thaw-boil cycle of the cell-containing sample fluid 120 may be implemented, which promotes cell lysis to occur therein.

Additionally, sample fluid 120 may contain certain beads (not shown) for interacting with the cells during the thermal cycling process in any manner that promotes cell lysis. In another embodiment, thermoelectric module 3810 may provide both the cooling and heating function and, thus, be used without a laser source for heating.

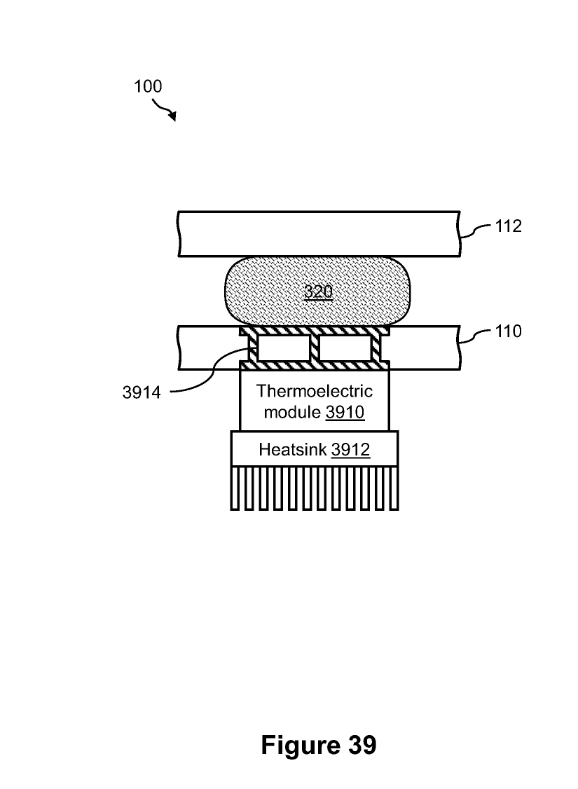

FIG. 39 illustrates yet another cross-sectional view of a portion of droplet actuator 100 of FIG. 1 and shows an example of using thermal cycling for promoting cell lysis. In this example, the combination of a thermoelectric module 3910 and a heat sink 3912 is in thermal contact with the outer surface of bottom substrate 110 of droplet actuator 100. In one example, thermoelectric module 3910 is a Peltier cooler and heat sink 3912 is an air cooled heat sink. Thermoelectric module 3910 is capable of providing both the cooling and heating. Alternatively, the combination of a thermoelectric module 3910 and a heat sink 3912 may be in thermal contact with the outer surface of top substrate 112 of droplet actuator 100.

A thermal conduction structure 3914 is incorporated into bottom substrate 110 of droplet actuator 100. Thermal conduction structure 3914 is provided in order to transfer the thermal energy from thermoelectric module 3910 to, for example, a cell-containing sample droplet 320 in gap 114 of droplet actuator 100. Therefore, thermal conduction structure 3914 may be any structure that is formed of any thermally conductive material, such as, but not limited to, aluminum and copper. Alternatively, thermal conduction structure 3914 may be incorporated into top substrate 112 of droplet actuator 100.

In another example, a thermoelectric module 3810 is in thermal contact with a substrate of droplet actuator 100, for example bottom substrate 110. In one example, thermoelectric module 3810 may include a Peltier cooler. Additionally, a heat source, such as, but not limited to, laser source 1310 of FIG. 13 and/or laser source 2610 of FIG. 26, may be positioned at an opposing substrate, of droplet actuator 100, for example top substrate 112, for emitting laser energy to a cell-containing sample droplet positioned at a certain droplet operations electrode 116. In this example, top substrate 112 is substantially transparent to laser energy. Thermoelectric module 3810 is the cooling source, while laser source 1310 is the heat source.

By controlling the cooling and heating operations of thermoelectric module 3910, a freeze-thaw cycle of the cell-containing sample droplet 320 may be implemented, which promotes cell lysis to occur therein. Additionally, sample droplet 320 may contain certain beads (not shown) for interacting with the cells during the thermal cycling process in any manner that promotes cell lysis.

### 7.9 Cell Lysis by Dounce Homogenizer

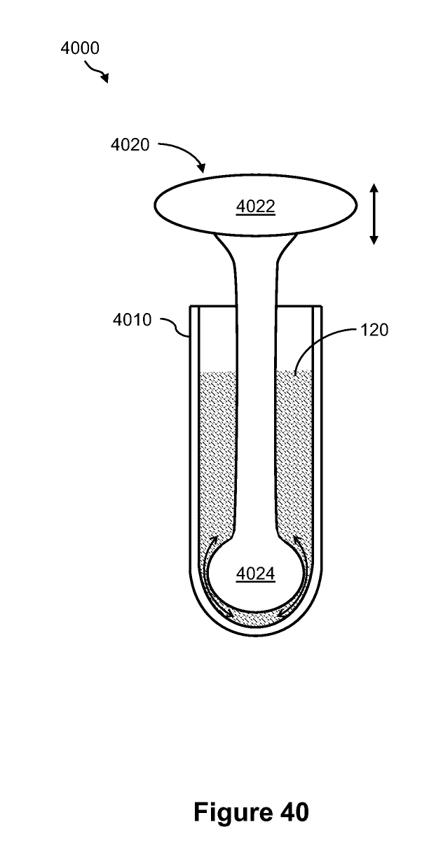

FIG. 40 illustrates a cross-sectional view of a Dounce homogenizer 4000 that may be used for promoting cell lysis in a cell-containing sample fluid by mechanical shearing. Dounce homogenizer 4000 may be, for example, any standard Dounce homogenizer. Dounce homogenizer 4000 may include a vessel or tube 4010 and a pestle 4020 of sufficient size. Vessel or tube 4010 and pestle 4020 may be formed, for example, of glass or plastic, or other suitable material. Pestle 4020 may include a handle 4022 and a rounded tip 4024 that is designed to be tightly fitted into the bottom of vessel or tube 4010. Vessel or tube 4010 may contain a quantity of cell-containing sample fluid 120. Pestle 4020 is manually manipulated up and down within vessel or tube 4010. In doing so, mechanical shearing of the cells takes place between tip 4024 of pestle 4020 and the walls of vessel or tube 4010, thereby promoting cell lysis in sample fluid 120. In one embodiment, a Dounce homogenizer is integrated with a substrate of the droplet actuator. Following homogenization, homogenized liquid is flowed from the homogenizer into a droplet operations gap of the droplet actuator where the liquid may be subjected to one or more droplet operations.

### 7.10 Systems

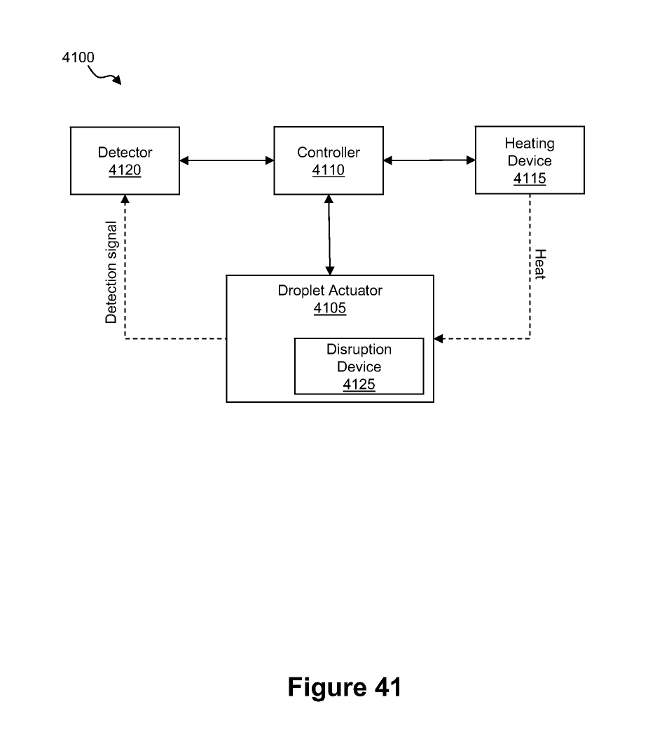

As illustrated in FIG. 41, the invention may include a system 4100 including a droplet actuator 4105 and a controller 4110 electrically coupled to droplet actuator 4105, a heating device 4115, and a detector 4120, and any other input and/or output devices (not shown), wherein the controller controls the overall operation of the system. Controller 4110 may, for example, be a general purpose computer, special purpose computer, personal computer, or other programmable data processing apparatus. Controller 4110 serves to provide processing capabilities, such as storing, interpreting, and/or executing software instructions, as well as controlling the overall operation of the system, and is electronically coupled to various hardware components of the invention, such as droplet actuator 4105, detector 4120, heating device 4115, and any input and/or output devices. Controller 4110 may be configured and programmed to control data and/or power aspects of these devices. For example, in one aspect, with respect to droplet actuator 4105, controller 4110 controls droplet manipulation by activating/deactivating electrodes.

In one example, heating device 4115 may be heater bars that are positioned in relation to droplet actuator 4105 for providing thermal control thereof.

In one example, detector 4120 may be an imaging system that is positioned in relation to droplet actuator 4105. In one example, the imaging system may include one or more light-emitting diodes (LEDs) (i.e., an illumination source) and a digital image capture device, such as a charge-coupled device (CCD) camera.

Droplet actuator 4105 may include disruption device 4125. Disruption device 4125 may include any device that promotes disruption (lysis) of materials, such as tissues, cells and spores in a droplet actuator. Disruption device 4125 may, for example, be a sonication mechanisms, heating mechanisms, mechanical shearing mechanisms, bead beating mechanisms, physical features incorporated into the droplet actuator 4105, electric field generating mechanism, thermal cycling mechanism, or a combination of two or more of the above. Disruption device 4125 may be controlled by controller 4110.

Referring to FIGS. 1 through 41, the invention may be embodied as a method, system, or computer program product. Aspects of the invention may take the form of hardware embodiments, software embodiments (including firmware, resident software, micro-code, etc.), or embodiments combining software and hardware aspects that may all generally be referred to herein as a “circuit,” “module” or “system.” Furthermore, the methods of the invention may take the form of a computer program product on a computer-usable storage medium having computer-usable program code embodied in the medium.

Any suitable computer useable medium may be utilized for software aspects of the invention. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium would include some or all of the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission medium such as those supporting the Internet or an intranet, or a magnetic storage device. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device.

Computer program code for carrying out operations of the invention may be written in an object oriented programming language such as Java, Smalltalk, C++ or the like. However, the computer program code for carrying out operations of the invention may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network (LAN) or a wide area network (WAN), or the connection may be made to an external computer (for example, through the Internet using an Internet Service Provider).

Certain aspects of invention are described with reference to various methods and method steps. It will be understood that each method step can be implemented and controlled by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the methods.

The computer program instructions may also be stored in a computer-readable memory that can direct a computer or other programmable data processing apparatus to function in a particular manner, such that the instructions stored in the computer-readable memory produce an article of manufacture including instruction means which implement various aspects of the method steps.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing various functions/acts specified in the methods of the invention.

### 8 CONCLUDING REMARKS

The foregoing detailed description of embodiments refers to the accompanying drawings, which illustrate specific embodiments of the invention. Other embodiments having different structures and operations do not depart from the scope of the present invention.

The term “the invention” or the like is used with reference to certain specific examples of the many alternative aspects or embodiments of the applicants' invention set forth in this specification, and neither its use nor its absence is intended to limit the scope of the applicants' invention or the scope of the claims. This specification is divided into sections for the convenience of the reader only. Headings should not be construed as limiting of the scope of the invention. The definitions are intended as a part of the description of the invention. It will be understood that various details of the present invention may be changed without departing from the scope of the present invention. Furthermore, the foregoing description is for the purpose of illustration only, and not for the purpose of limitation.

1.-49. (canceled)

50. A droplet actuator for conducing droplet operations, comprising:
(a) a bottom substrate and a top substrate separated from each other to form a gap;
(b) an arrangement of droplet operations electrodes on at least one of the bottom and top substrate for conducting droplet operations;
(c) a sample supply for supplying a quantity of sample fluid containing cells to be lysed into the gap; and
(d) a cell disruption device for disrupting and lysing cells in the sample fluid.

51. The droplet actuator of any of claims 50 and following, wherein the cell disruption device is an ultrasonic device, and particles in cell-containing sample droplets to be activated by the ultrasonic device to cause cavitation in the sample droplets.

52. The droplet actuator of any of claims 51 and following, further comprising rough features on the gap facing surface of one or both substrates.

53. The droplet actuator of any of claims 50 and following, further comprising a barrier for retaining microemulsion droplets that may result from cell disruption.

54. The droplet actuator of any of claims 50 and following, wherein the cell disruption device is an electric field generator.

55. The droplet actuator of any of claims 54 and following, wherein the electric field generator comprises electrodes.

56. The droplet actuator of any of claims 50 and following, wherein the cell description device comprises at least one pair of field generating electrodes arranged to have a droplet in contact therewith at opposing sides of the droplet.

57. The droplet actuator of any of claims 56 and following, further comprising a droplet operations having a clearance region, and arranged between the field generating electrodes.

58. The droplet actuator of any of claims 55 and following, further comprising a sample reservoir, and the electrodes are located in, or in proximity to, the sample reservoir.

59. The droplet actuator of any of claims 55 and following, wherein the electrodes are located in the gap, with dielectric layers on the top substrate and the bottom substrate.

60. The droplet actuator of any of claims 55 and following, wherein the electrodes are on the same substrate spaced from each other to contact opposite edges of a droplet.

61. The droplet actuator of any of claims 55 and following, wherein the electrodes are specially configured droplet operations electrodes.

62. The droplet actuator of any of claims 55 and following, wherein the electrodes comprise an array of electrodes extending into the gap to cause a disruptive electric field.

63. The droplet actuator of any of claims 62 and following, wherein the electrodes are electroporation electrodes arranged alongside the droplet operations electrodes.

64. The droplet actuator of any of claims 63 and following, wherein the electroporation electrodes have clearance regions.

65. The droplet actuator of any of claims 64 and following, wherein the electroporation electrodes are implemented by solder posts.

66. The droplet actuator of any of claims 58 and following, further comprising a laser source directed at cell-containing sample fluid in the sample reservoir.

67. The droplet actuator of any of claims 50 and following, wherein the cell disruption device is a Dounce homogenizer.

68. The droplet actuator of any of claims 67 and following, wherein the Dounce homogenizer is integral with a substrate of the actuator.
